(19) United States
(12) Patent Application Publication
(10) Pub. No.: US 2023/0330040 A1
(43) Pub. Date: Oct. 19, 2023

(54) IMMUNOLOGICAL EFFECTS OF METABOLITES

(71) Applicant: The Regents of the University of California, Oakland, CA (US)

(72) Inventors: Angela ZIVKOVIC, St. Helena, CA (US); Christopher RHODES, Davis, CA (US)

(21) Appl. No.: 18/027,255

(22) PCT Filed: Sep. 20, 2021
(86) PCT No.: PCT/US2021/051104

§ 371 Date: Mar. 20, 2023

Related U.S. Application Data
(60) Provisional application No. 63/081,205, filed on Sep. 21, 2020.

(51) Int. Cl. A61K 31/132; A61K 31/164; A61K 31/455

(52) U.S. Cl. CPC A61K 31/132; A61K 31/164; A61K 31/455

(57) ABSTRACT

The present disclosure provides compositions and methods of using one or more of the metabolites spermidine, palmitoylethanolamide (PEA), oleoylethanolamide (OEA), and 1-methylnicotinamide (1-MNA) to induce an anti-inflammatory, anti-oxidant, and/or immuno-modulatory effect in a subject. The compositions and methods described herein can enhance biochemical functionalities relevant to overall health and disease progression, promote longevity and healthspan, and/or delay or inhibit the cellular aging process in the subject.

### CROSS-REFERENCE TO RELATED PATENT APPLICATIONS

The present patent application claims benefit of priority to U.S. Provisional Patent Application No. 63/081,205, filed Sep. 21, 2020, which is incorporated by reference for all purposes.

### BACKGROUND OF THE DISCLOSURE

Prolonged Fasting has consistently been shown to induce a broad range of functional biochemical benefits in both model organisms and human trials including anti-cancer, anti-inflammatory; cardioprotective, anti-oxidant, stem and immune cell regeneration, and longevity enhancing effects. Despite the wealth of documented benefits of prolonged fasting, the full scope of the biochemical effects of fasting as well as the precise mediators and mechanisms behind these effects remain largely unstudied in humans.

### SUMMARY OF THE DISCLOSURE

In one aspect, the disclosure provides a composition comprising one or more of metabolites selected from the group consisting of spermidine, 1-methylnicotinamide (1-MNA), palmitoylethanolamide (PEA), and oleoylethanolamide (OEA) in an amount sufficient to induce an anti-inflammatory, anti-oxidant, and/or immuno-modulatory effect in a subject. In one aspect, the disclosure provides a composition comprising one or more of metabolites selected from the group consisting of spermidine, 1-methylnicotinamide (1-MNA), palmitoylethanolamide (PEA), and oleoylethanolamide (OEA) in an amount sufficient to elevate the circulating levels of the metabolites to a same level or higher than the circulating levels of the metabolites that are achieved through prolonged (e.g., at least 20 hours, e.g., 20-72 hours) fasting in a subject. In some embodiments, the composition comprises two of the metabolites (e.g., spermidine and 1-MNA, spermidine and PEA, spermidine and OEA, 1-MNA and PEA, 1-MNA and OEA, or PEA and OEA). In some embodiments, the composition comprises three of the metabolites (e.g., 1) sperimidine, 1-MNA, and PEA, 2) spermidine, 1-MNA, and OEA, 3) spermidine, PEA, and OEA, or 4) 1-MNA, PEA, and OEA). In some embodiments, the composition comprises all four of the metabolites.

In one aspect, the disclosure provides a composition comprising one or more of metabolites selected from the group consisting of spermidine or a precursor thereof, 1-methylnicotinamide (1-MNA) or a precursor thereof, palmitoylethanolamide (PEA) or a precursor thereof, and oleoylethanolamide (OEA) or a precursor thereof in an amount sufficient to induce an anti-inflammatory, anti-oxidant, and/or immuno-modulatory effect in a subject. In one aspect, the disclosure provides a composition comprising one or more of metabolites selected from the group consisting of spermidine or a precursor thereof, 1-methylnicotinamide (1-MNA) or a precursor thereof, palmitoylethanolamide (PEA) or a precursor thereof, and oleoylethanolamide (OEA) or a precursor thereof in an amount sufficient to elevate the circulating levels of the metabolites to a same level or higher than the circulating levels of the metabolites that are achieved through prolonged (e.g., at least 20 hours, e.g., 20-72 hours) fasting in a subject. In some embodiments, the composition comprises two of the metabolites (e.g., spermidine or a precursor thereof and 1-MNA or a precursor thereof, spermidine or a precursor thereof and PEA or a precursor thereof, spermidine or a precursor thereof and OEA or a precursor thereof, 1-MNA or a precursor thereof and PEA or a precursor thereof, 1-MNA or a precursor thereof and OEA or a precursor thereof, or PEA or a precursor thereof and OEA or a precursor thereof). In some embodiments, the composition comprises three of the metabolites (e.g., 1) sperimidine or a precursor thereof, 1-MNA or a precursor thereof, and PEA or a precursor thereof, 2) spermidine or a precursor thereof, 1-MNA or a precursor thereof, and OEA or a precursor thereof, 3) spermidine or a precursor thereof, PEA or a precursor thereof, and OEA or a precursor thereof, or 4) 1-MNA or a precursor thereof, PEA or a precursor thereof, and OEA or a precursor thereof). In some embodiments, the composition comprises all four of the metabolites.

In some embodiments, the composition comprises a precurser of 1-MNA selected from the group consisting of nictotinamide, niacinamide, and nicotinamide riboside. In some embodiments, the composition comprises a precurser of PEA is palmitic acid. In some embodiments, the composition comprises a precurser of OEA is oleic acid.

In some embodiments of this aspect, the ratio of two, three, or four of the metabolites is about 10000:1000:1:1000 of spermidine:1 MNA:PEA:OEA (w:w:w:w). In some embodiments, the composition comprises 5-15 mg spermidine, 400-1200 mg PEA, 300-600 mg OEA, and 500-1000 mg nicotinamide.

In some embodiments, the composition is formulated as a dietary supplement. In certain embodiments, the composition is formulated for oral administration. For example, the composition can be formulated as a pill, a tablet, a powder, a solid food coating gummy, sublingual, spray, candy, nutrition bar, energy shot, beverage, or a syrup. In some embodiments, the composition is formulated for intravenous administration, transdermal administration, sublingual administration, or topical administration.

In another aspect, the disclosure features a method for inducing an anti-inflammatory, anti-oxidant, and/or immuno-modulatory effect in a subject, comprising administering to the subject one or more of metabolites selected from the group consisting of spermidine, 1-methylnicotinamide (1-MNA), palmitoylethanolamide (PEA), and oleoylethanolamide (OEA) in an amount sufficient to induce the anti-inflammatory, anti-oxidant, and/or immuno-modulatory effect in the subject. In another aspect, the disclosure features a method for elevating the circulating levels of the metabolites to a same level or higher than the circulating levels of the metabolites that are achieved through prolonged (e.g., at least 20 hours, e.g., 20-72 hours) fasting in a subject, comprising administering to the subject one or more of metabolites selected from the group consisting of spermidine. 1-methylnicotinamide (1-MNA), palmitoylethanolamide (PEA), and oleoylethanolamide (OEA) in an amount sufficient to induce elevate the circulating levels of the metabolites to a same level or higher than the circulating levels of the metabolites that are achieved through prolonged (e.g., at least 20 hours, e.g., 20-72 hours) fasting in a subject.

In some embodiments, the method decreases the amount of tumor necrosis factor alpha (TNF-α) secreted by macrophages in the subject relative to the amount of TNF-α, secreted by macrophages in the subject prior to the subject receiving the metabolite. In certain embodiments, the amount of TNF-α secreted by macrophages after the subject receiving the metabolite is less than 90% (e.g., less than 85%, less than 80%, less than 75%, less than 70%, less than 65%, less than 60%, less than 55%, less than 50%, less than 45%, less than 40%, less than 35%, less than 30%, less than 25%, less than 20%, less than 10%, or less than 5%) of the amount of TNF-α secreted by macrophages prior to the subject receiving the metabolite.

In some embodiments of this aspect, the method increases total antioxidant capacity of the subject's plasma relative to the total antioxidant capacity of the subject's plasma prior to the subject receiving the metabolite. In some embodiments, the method increases cholesterol efflux of the subject relative to the cholesterol efflux of the subject prior to the subject receiving the metabolite.

In some embodiments, the method decreases the amount of reactive oxygen species (ROS) produced by macrophages in the subject relative to the amount of ROS produced by macrophages in the subject prior to the subject receiving the metabolite. In some embodiments, the method decreases cyclooxygenase (COX) activity in macrophages of the subject relative to the COX activity in macrophages of the subject prior to the subject receiving the metabolite. In some embodiments, the method modulates the phenotype of macrophages away from a pro-inflammatory and toward a pro-resolving phenotype (measured as decreased nitric oxide synthase (NOS) activity in macrophages of the subject relative to the NOS activity in macrophages of the subject prior to the subject receiving the metabolite).

In some embodiments, the method decreases macrophage M1 polarization and/or increases M2 polarization (measured as decreased nitric oxide synthase (NOS) activity in macrophages of the subject relative to the NOS activity in macrophages of the subject prior to the subject receiving the metabolite and/or measured as increased arginase activity in the macrophages of the subject relative to the arginase activity in the macrophages of the subject prior to the subject receiving the metabolite) in the subject relative to the macrophage polarization in the subject prior to the subject receiving the metabolite.

In some embodiments, the method increases arginase activity in the subject relative to the arginase activity in the subject prior to the subject receiving the metabolite.

In some embodiments, the method increases macrophage M2 polarization in the subject relative to the macrophage M2 polarization in the subject prior to the subject receiving the metabolite.

In other embodiments, the method extends longevity and/or improves cognitive and/or physical performance of the subject.

In some embodiments, the subject is on a fasting diet. In certain embodiments, when the subject is on a fasting diet (e.g., fasting for more than 12 hours (e.g., between 12 and 15 hours, between 15 and 20 hours, between 20 and 25 hours, between 25 and 30 hours, between 30 and 36 hours, or more than 36 hours)), one or more of metabolies selected from the group consisting of pentose acid, indolepropionate, gentisate, piperine, and hydrocinnamate is substantially depleted in the subject.

In some embodiments, the subject has an inflammatory disorder. In some embodiments, the inflammatory disorder is selected from the group consisting of rheumatoid arthritis (RA), systemic lupus erythematosus (SLE), ANCA-associated vasculitis, antiphospholipid antibody syndrome, autoimmune hemolytic anemia, chronic inflammatory demyelinating neuropathy, graft-vs-host disease (GVHD), dermatomyositis, Goodpasture's Syndrome, organ system-targeted type II hypersensitivity syndromes, Guillain Barre syndrome, chronic inflammatory demyelinating polyneuropathy (CIDP), dermatomyositis, Felty's syndrome, autoimmune thyroid disease, ulcerative colitis, autoimmune liver disease, idiopathic thrombocytopenia purpura, Myasthenia Gravis, neuromyelitis optica, pemphigus, Sjogren's Syndrome, autoimmune cytopenias, synovitis, dermatomyositis, systemic vasculitis, glomerulitis, irritable bowel syndrome (IBS), and vasculitis. In some embodiments, the subject has a metabolic disorder. In some embodiments the metabolic disorder is selected from the group consisting of non-alcoholic fatty liver disease (NAFLD), non-alcoholic steatohepatitis (NASH), diabetes, and metabolic syndrome. In certain embodiments, the subject is overweight.

In certain embodiments, the metabolite is administered to the subject one or more times daily. In certain embodiments, the metabolite is administered to the subject during food intake. In certain embodiments, the metabolite is administered to the subject after at least 5 hours (e.g., between 5 and 10 hours, between 10 and 15 hours, between 15 and 20 hours, between 20 and 25 hours, between 25 and 30 hours, between 30 and 36 hours, or more than 36 hours) of fasting.

In some embodiments, the method comprises administering two of the metabolites (e.g., spermidine or a precursor thereof and 1-MNA or a precursor thereof, spermidine or a precursor thereof and PEA or a precursor thereof, spermidine or a precursor thereof and OEA or a precursor thereof, 1-MNA or a precursor thereof and PEA or a precursor thereof, 1-MNA or a precursor thereof and OEA or a precursor thereof, or PEA or a precursor thereof and OEA or a precursor thereof). In some embodiments, the method comprises administering three of the metabolites (e.g., 1) sperimidine or a precursor thereof, 1-MNA or a precursor thereof, and PEA or a precursor thereof, 2) spermidine or a precursor thereof, 1-MNA or a precursor thereof, and OEA or a precursor thereof, 3) spermidine or a precursor thereof, PEA or a precursor thereof, and OEA or a precursor thereof, or 4) 1-MNA or a precursor thereof, PEA or a precursor thereof, and OEA or a precursor thereof). In certain embodiments, the method comprises administering all four of the metabolites or a precursor thereof. In particular embodiments of the method, the subject is a human.

Also provided is a method extending lifespan, healthspan, healthy aging, altering biochemical pathways associated with the aging process or the treatment or prevention of age related diseases (including but not limited to frailty, sarcopenia, dementia, Alzheimers disease, cognitive decline, cancer, or arthritis) in a subject in need thereof comprising administering to the subject a therapeutically effective amount of the composition as described above or elsewhere herein. In some embodiments, the subject is a human.

Also provided is a method of increasing plasma cholesterol efflux ability in a subject in need thereof comprising administering to the subject a therapeutically effective amount of the composition as described above or elsewhere herein. In some embodiments, the subject is at risk for or has heart disease, stroke, arteral plaque formation or other cardiovascular disease risk factors. In some embodiments, the subject is a human.

### BRIEF DESCRIPTION OF THE DRAWINGS

FIG. 1: Timeline of 3-day Human Fasting Trial: 20 Participants underwent a 3-day clinical trial consisting of 4 study visits in 4 distinct nutritional states to allow for the sensitive assessment of the effects of 36 hrs of fasting versus an overnight fasted state and the carryover effects of fasting onto the next eating day. On Day 1, participants provided an overnight fasting Baseline blood sample (A) then went about their normal routine and habitual diet while tracking their food intake until 6 PM where participants ate their last meal. A 2 hr postprandial Fed blood sample was taken at 8 PM on Day 1 (B) after which, participants began their 36 hr fast while being monitored for compliance via glucose monitors. At 8 AM on Day 3, participants provided a 36 hr Fasted blood draw (C) and then were given a copy of their diet record from Day 1 and instructed to eat the identical diet as they recorded on Day 1. At 6 PM on Day 3, participants ate their last meal and received a final 2 hr postprandial Refed blood draw (D) concluding the study.

FIG. 2: CONSORT Diagram Indicating the Structure of the 36 hr Fasting Trial: 72 interested individuals were screened for eligibility and 52 were excluded based on inclusion and exclusion criteria (Supplemental Information). A final 20 participants (10 men and 10 women) were enrolled in the study and underwent the 3-day trial consisting of the intervention of 36 hrs of fasting. There were no adverse events, dropouts, or participants lost to follow-up. All 20 participants completed the trial successfully with no protocol violations and were all included in experimental analysis.

FIGS. 3A-3G: In vitro Analyses of Participant Plasma Functionality and the Effects of Participant Plasma Treatment on Human Macrophage Functionality. A) In vitro analysis of the total antioxidant capacity of participants plasma showing significant differences between the Baseline and Fasted states (p<0.0001) and the Fed and Refed states (p=0.034). B) In vitro analysis of the cholesterol efflux capacity of participant plasma from lipid-loaded primary human macrophage showing significant differences between the Baseline and Fasted states (p<0.0001) and the Fed and Refed states (p<0.0001). C) In vitro analysis of intracellular reactive oxygen species (ROS) production in hydrogen peroxide stimulated primary human macrophage treated with participant plasma showing significant differences between the Baseline and Fasted states (p=0.0018). D) In vitro analysis of tumor necrosis factor alpha (TNF-α) secretion from citrullinated fibrinogen immune complex (cFb ICs) stimulated primary human macrophage treated with participant plasma showing significant differences between the Baseline and Fasted states (p<0.001) and the Fed and Refed states (p=0.0004). E) In vitro analysis of total cyclooxygenase (COX) activity in lipopolysaccharide (LPS) stimulated THP-1 macrophage treated with participant plasma showing significant differences between the Baseline and Fasted states (p=0.043) and the Fed and Refed states (p=0.034). F) In vitro analysis of nitric oxide synthase (NOS) activity in LPS and interferon gamma (INF-γ) stimulated THP-1 macrophage treated with participant plasma showing significant differences between the Baseline and Fasted states (p=0.042) and the Fed and Refed states (p=0.017). G) In vitro analysis of arginase activity in LPS and INF-γ treated THP-1 macrophage treated with participant plasma showing significant differences between the Baseline and Fasted states (p<0.0001).

FIGS. 4A-4J: Metabolomic Analysis of Participant Plasma and Upregulated Immunomodulatory Metabolites During Fasting. A) Pathway analysis of metabolic datasets showing significantly differentially regulated pathways between the Baseline and Fasted State. B) Circulating levels of the ketone body beta-hydroxybutyrate (BHB) in participant plasma showing significant differences between the Baseline and Fasted states (p=1.31e-18). C) Circulating levels of (R)-3-hydroxybutyrylcarnitine in participant plasma, the most significantly altered metabolite between the Baseline and Fasted state, showing significant differences between the Baseline and Fasted states (p=2.53e-21) and the Fed and Refed states (p=8.03e-9). D) Circulating levels of the ketone body acetoacetate in participant plasma showing significant differences between the Baseline and Fasted states (p=2.44e-21) and the Fed and Refed states (p=0.025). E) Circulating levels of the immunomodulatory metabolite spermidine in participant plasma showing significant differences between the Baseline and Fasted states (p=0.0001) and the Fed and Refed states (p=0.025). F) Circulating levels of the immunomodulatory metabolite 1-methlynicotinamide (1-MNA) in participant plasma showing significant differences between the Baseline and Fasted states (p=0.0038). G) Circulating levels of the immunomodulatory palmitoylethanolamide (PEA) in participant plasma showing significant differences between the Baseline and Fasted states (p=9.271e-11). H) Circulating levels of the immunomodulatory metabolite oleoylethanolamide (OEA) in participant plasma showing significant differences between the Baseline and Fasted states (p=1.05e-5). H) Circulating levels of the immunomodulatory metabolite oleoylethanolamide (OEA) in participant plasma showing significant differences between the Baseline and Fasted states (p=1.05e-5). I) Principal Component Analysis of complete metabolite datasets between Baseline, Fed, Fasted and Refed states showing large differentiation the Fasted states in comparison to the three other states. Timepoint A: Baseline state; B: Fasted state; C: Fed state; and D: Refed state. J) Volcano Plot Analysis of Fasted metabolite levels in comparison to Baseline metabolite levels showing over 375 significantly up- or down-regulated metabolites between the two states.

FIGS. 5A-5G: Anti-inflammatory Functionalities and C. elegans Lifespan Extending Effects of Immunomodulatory Fasting Metabolites: A) Tumor necrosis factor alpha (TNF-α) secretion from citrullinated fibrinogen immune complex (cFb ICs) stimulated primary human macrophage treated with beta-hydroxybutyrate (BHB), spermidine, 1-methylnicotinamide (1-MNA), palmitoylethanolamide (PEA), and oleoylethanolamide (OEA) at a final concentration of 1 mM-10 nM. TNF-α levels from unstimulated macrophage are shown as negative control values and TNF-α levels from stimulated macrophage without any other treatment are shown as positive control values. B) TNF-α secretion from cFB IC stimulated primary human macrophage treated with spermidine (100 μM), 1-MNA (100 μM), PEA (10 nM), OEA (10 μM), and a combination treatment (Combo) of all four metabolites at their individual concentrations. These concentrations were used for all vitro analyses (B-F). Negative and positive control values for all in vitro metabolite analyses (B-F) were generated from unstimulated macrophage or stimulated macrophage with no other treatment respectively as above. Significance between treatments and controls for all in vitro analyses (B-F) are given as: a) significantly different from Combo treatment (p<0.05), b) significantly different from positive control (p<0.05), c) significantly different from the negative control (p<0.05) C) Intracellular reactive oxygen species (ROS) production from hydrogen peroxide stimulated primary human macrophage treated with spermidine, 1-MNA, PEA, OEA, and Combo. D) Total cyclooxygenase (COX) activity from lipopolysaccharide (LPS) stimulated THP-1 macrophage treated with spermidine, 1-MNA, PEA, OEA, and Combo. E) Total nitric oxide synthase (NOS) activity from LPS and interferon gamma (INF-γ) stimulated THP-1 macrophage treated with spermidine, 1-MNA, PEA, OEA, and Combo. F) Arginase activity from LPS and INF-γ stimulated THP-1 macrophage treated with spermidine, 1-MNA, PEA, OEA, and Combo. G) Lifespan analysis of C. elegans with either no treatment (Control) or lifelong exposure to spermidine (100 μM), 1-MNA (100 μM), and PEA at either 100 μM or 10 μM. Significant lifespan extension was observed for all metabolites except 1-MNA and the greatest lifespan extension was observed for the combination treatment of all four metabolites; spermidine (p=0.0064), PEA 100 μM (p<0.0001), PEA 10 μM (p<0.0001), OEA (p<0.0001), Combination (p<0.0001).

FIGS. 6A-6E: Pentose acid, indolepropionate, piperine, gentisate, and hydrocinnamate were substantially depleted during prolonged fasting. Timepoint A: Baseline state; B: Fasted state; C: Fed state; and D: Refed state.

FIG. 7A-D: Subject plasma anti-inflammatory ability as measured by In vitro analysis of tumor necrosis factor alpha (TNF-α) secretion from citrullinated fibrinogen immune complex (cFb ICs) stimulated primary human macrophage treated with participant plasma. A) Low Dose supplementation arm showing non-significant differences between the T0 and T1 timepoint and significantly lower TNF-α secretion between the T0 and T2 timepoint. B) Medium Dose supplementation arm showing non-significant differences between the T0 and T1 timepoint and significantly lower TNF-α secretion between the T0 and T2 timepoint. High Dose supplementation arm showing non-significant differences between the T0 and T1 timepoint and significantly lower TNF-α secretion between the T0 and T2 timepoint. D) Control arm showing significant increase in TNF-α secretion between the T0 and T1 timepoint and non-significant differences between the T0 and T2 timepoint.

FIG. 8A-D: Subject plasma antioxidant ability as measured by in vitro of the accumulation of intracellular reactive oxygen species (ROS) in primary human macrophage treated with the pro-oxidant tert-Butyl hydroperoxide (TBHP) along with participant plasma. A) Low Dose supplementation arm showing significantly lower intracellular ROS in the T1 and T2 timepoints vs the T0 timepoint. B) Medium Dose supplementation arm showing significantly lower intracellular ROS in the T1, T2, and T4 timepoints vs the T0 timepoint. C) High Dose supplementation arm showing significantly lower intracellular ROS in the T1, T2, and T4 timepoints vs the T0 timepoint. D) Control arm showing non-significant differences between any timepoint.

FIG. 9A-D: Subject plasma cholesterol efflux ability from lipid-loaded primary human macrophage exposed to participant plasma. A) Low Dose supplementation arm showing non-significant differences between all timepoints. B) Medium Dose supplementation arm showing non-significant differences between all timepoints. C) High Dose supplementation arm showing significant increases in percent cholesterol efflux in the T1 vs T0 timepoint. D) Control arm showing significant decreases in percent cholesterol efflux in the T1 vs T0 timepoint.

### DETAILED DESCRIPTION OF THE DISCLOSURE

### I. Introduction

The present disclosure investigates the effects of 36 hours of a water-only fast on the functionality of human plasma isolated from 20 young healthy male and female subjects (Age: 20-40, Male:10, Female: 10, BMI: 17-25). The study showed that prolonged fasting induces stark improvements in the biochemical functionalities of human plasma. The effects on the human plasma are due, at least in part, to numerous naturally occurring endogenous metabolites whose plasma concentrations are significantly upregulated during fasting as identified by a comprehensive metabolic panel of subject plasma samples. For example, in the study, 36 hours of fasting significantly increases the ability of subject plasma to promote cholesterol efflux from cholesterol-loaded THP-1 monocytes by up to 25% and significantly increases the ability of subject plasma to suppress TNF-α secretion from primary macrophage stimulated with pro-inflammatory citrullinated-fibrinogen immune complexes by up to 62%.

Furthermore, using a comprehensive metabolomic panel, the present disclosure identified compounds that are significantly upregulated during fasting: spermidine, palmitoylethanolamide (PEA), oleoylethanolamide (OEA), and 1-methylnicotinamide (1-MNA). The present disclosure is directed to the compositions and methods of using any of these compounds alone or in combination to elicit the beneficial biological effects of fasting, enhance biochemical functionalities relevant to overall health and disease progression, promote longevity and healthspan, and/or delay or inhibit the cellular aging process.

### II. Definitions

As used herein, the term “fasting diet” refers to a diet that has at least 5 hours (e.g., at least 6, 8, 10, 12, 14, 16, 18, 20, 22, 24, 26, 28, 30, 32, 34, 36, 38, 40, or 42 hours) in between consumption of any food. In some embodiments, a “prolonged fasting diet” refers to a diet that has at least 24 hours (e.g., 26, 28, 30, 32, 34, 36, 38, 40, or 42 hours) in between consumption of any food.

As used herein, the term “metabolic disorder” refers to a disease, disorder, or syndrome that is related to a subject's metabolism, such as breaking down carbohydrates, proteins, and fats in food to release energy, and converting chemicals into other substances and transporting them inside cells for energy utilization and/or storage. Some symptoms of a metabolic disease include high serum triglycerides, high low-density cholesterol (LDL), low high-density cholesterol (HDL), and/or high fasting insulin levels, elevated fasting plasma glucose, abdominal (central) obesity, and elevated blood pressure. In the present invention, metabolic diseases include, but are not limited to, obesity, Type-1 diabetes, and Type-2 diabetes.

As used herein, the term “inflammatory disorder” refers to a disease, disorder, or syndrome that is related to a subject's immune system. An inflammatory disorder generally involves an activation of the subject's immune system, either in responding to an illness or infection or, at other times, in attacking the body's own cells and tissues (e.g., autoimmune disorders).

As used herein, the term “overweight” is defined as a body mass index (BMI) of greater than 25 (or a BMI>23 in Asian populations), thus it includes pre-obesity defined as a BMI between 25 and 30 and obesity as defined by a BMI of 30 or more. Overweight can also be defined as having a waist circumference>35 inches for women and >40 inches for men.

As used herein, the term “total antioxidant capacity” refers to a measure that can be used to assess the antioxidant status of biological samples and can evaluate the antioxidant response against the free radicals produced in a given disease. Methods and techniques to measure total antioxidant capacity are available in the art, e.g., as described in Rubio et al., BMC Vet Res. 12:166, 2016 and Ialongo C. Review Clin Biochem 50(6):356-363, 2017.

As used herein, the term “cholesterol efflux” refers to a pathway transferring intracellular cholesterol from macrophages or other cells to extracellular acceptors such as apolipoprotein A-I (apoA-I) of high-density lipoprotein (HDL).

As used herein, the term “about” refers to a range of +/−10% For example, “about 100” is equivalent to “90 to 110.”

As used herein, the term “subject” refers to a mammal, e.g., preferably a human. Mammals include, but are not limited to, humans and domestic and farm animals, such as monkeys (e.g., a cynomolgus monkey), mice, dogs, cats, horses, pigs, and cows, livestock, etc.

A “precursor” of a metabolite described herein refers to a molecule, which when introduced into a subject, is metabolized into the metabolite via one or more molecular reactions.

“As used herein, “metabolite” refers to a chemical agent. Exemplary metabolites include spermidine, 1-methylnicotinamide (1-MNA), palmitoylethanolamide (PEA), and oleoylethanolamide (OEA).”

### III. Compositions

The present disclosure features compositions comprising one or more of metabolites selected from the group consisting of spermidine or a precursor thereof, 1-methylnicotinamide (1-MNA) or a precursor thereof, palmitoylethanolamide (PEA) or a precursor thereof, and oleoylethanolamide (OEA) or a precursor thereof in an amount sufficient to induce an anti-inflammatory, anti-oxidant, and/or anti-apoptotic effect in a subject. These compounds were identified to elicit beneficial functionalities of human plasma, mimicking the health effects observed after prolonged fasting, including cholesterol efflux capacity and altering the immunomodulatory capacity towards a more anti-inflammatory functional phenotype. Any one or more of the metabolites listed above can be replaced with a precurser thereof or a molecule (e.g., drug) that causes elevatation of levels of any of the metabolites in the subject's body.

Spermidine is a naturally occurring polyamine that has been shown to have anti-inflammatory; anti-proliferative, and significant longevity enhancing effects in multiple model organisms through its molecular activity as an autophagy inducer via its actions on the MAPK pathway. Exemplary precursers of spermidine include but are not limited to arginine, ornithine, and putrescine.

Palmitoylethanolamide (PEA) is an endogenous fatty acid mediator and stimulator of the PPAR-α pathway that has been shown to have potent anti-inflammatory and anti-athersclerotic activities as well as an ability to reduce pain, neuropathy, and neurodegenerative disease symptoms including Parkinson's disease and Alzheimer's disease. An exemplary precurser of PEA includes but is not limited to palmitic acid.

Oleoylethanolamide (OEA) is an endogenous fatty acid mediator and stimulator of the AMPK pathway that has been shown to act as an appetite suppressant and satiety regulator with both neuroprotective and anti-inflammatory abilities. An exemplary precurser of OEA includes but is not limited to oleic acid.

Finally, 1-methylnicotinamide (1-MNA) is an endogenous metabolite of nicotinamide shown to have wide ranging biochemical activities through multiple pathways including anti-cancer/anti-proliferative, anti-inflammatory and anti-thrombiotic activity via the COX-2/PGI2 pathway, and neuroprotective and anti-Alzheimers effects. Exemplary precursers of 1-MNA include but are not limited to niacin, nicotinamide, niacinamide, nicotinamide mononucleotide, and nicotinamide riboside.

In some embodiments, the composition comprises two of the metabolites or a precursor thereof selected from the group consisting of spermidine, 1-MNA, PEA, and OEA. For example, the composition can comprise: spermidine and 1-MNA, spermidine and PEA, spermidine and OEA, 1-MNA and PEA, 1-MNA and OEA, or PEA and OEA. In a composition comprising two metabolites or a precursor thereof, the amounts (e.g., by mole or weight) of the two metabolites can be the same or different. For example, the amount of spermidine to amount of 1-MNA (weight:weight) can be 15:1, 14:1, 13:1, 12:1, 11:1, 10:1, 9:1, 8:1, 7:1, 6:1, 5:1, 4:1, 3:1, 2:1, or 1:1, respectively. In some embodiments, the amount of spermidine to amount of PEA (weight:weight) can be 20,000:1, 15,000:1, 10,000:1, 8,000:1, 6,000:1, 4,000:1, 2,000:1, 1,000:1, 500:1, or 100:1, respectively. In some embodiments, the amount of spermidine to amount of OEA (weight:weight) can be 15:1, 14:1, 13:1, 12:1, 11:1, 10:1, 9:1, 8:1, 7:1, 6:1, 5:1, 4:1, 3:1, 2:1, or 1:1, respectively. In some embodiments, the amount of 1-MNA to amount of PEA can be 1,500:1, 1,200:1, 1,000:1, 800:1, 600:1, 400:1, 200:1, 100:1, 50:1, 30:1, 10:1, or 1:1, respectively. In some embodiments the amount of 1-MNA to amount of OEA (weight:weight) can be 10:1, 9:1, 8:1, 7:1, 6:1, 5:1, 4:1, 3:1, 2:1, 1:1, or 1:2, respectively. In some embodiments, the amount of PEA to amount of OEA (weight:weight) can be 1:1,500, 11,200, 1:1,000, 1:800, 1:600, 1:400, 1:200, 1:100, 150, 1:30, 1:20, 1:10, or 1:1, respectively.

In some embodiments, the composition comprises three of the metabolites or a precursor thereof selected from the group consisting of spermidine, 1-MNA, PEA, and OEA. For example, the composition can comprise: 1) sperimidine, 1-MNA, and PEA, 2) spermidine, 1-MNA, and OEA, 3) spermidine, PEA, and OEA, or 4) 1-MNA, PEA, and OEA. In a composition comprising three metabolites, the amounts (e.g., by mole or weight) of the three metabolites can be the same or different. For example, the amount of sperimidine to 1-MNA to PEA (weight:weight) can be 10000:1000:1, respectively. The amount of spermidine to 1-MNA to and OEA (weight:weight) can be 10:1:1, respectively. The amount of spermidine to PEA to OEA can be 10000:1:1000, respectively. The amount of 1-MNA to PEA to OEA (weight:weight) can be 1000:1:1000, respectively.

In some embodiments, the composition comprises all four of the metabolites spermidine, 1-MNA, PEA, and OEA or a precursor thereof. In some embodiments, the amount of spermidine to 1-MNA to PEA to OEA (weight:weight) is 10000:1000:1:1000, respectively. In some embodiments, the composition comprises 5-15 mg spermidine, 400-1200 mg PEA, 300-600 mg OEA, and 500-1000 mg nicotinamide, which is optionally administered daily. For example, in some embodiments, the composition comprises 5 mg spermidine, 400 mg PEA, 300 mg OEA, and 500 mg nicotinamide, or alternatively 15 mg spermidine, 1200 mg PEA, 600 mg OEA, and 1000 mg nicotinamide.

The compositions disclosed herein comprising one or more of spermidine, 1-MNA, PEA, and OEA or a precursor thereof can be administered orally as a dietary supplement. For example, the composition can be formulated as one or more pills, one or more tablets, or one or more bottles of syrup. In some embodiments, the compositions can be administered to a subject who is on a prolonged fasting diet, which refers to a diet that has greater than 24 hours in between meals.

### IV. Methods

The present disclosure also features methods for inducing an anti-inflammatory, anti-oxidant, and/or immuno-modulatory effect in a subject by administering to the subject one or more of metabolites selected from the group consisting of spermidine, 1-MNA, PEA, and OEA or a precursor thereof in an amount sufficient to induce the anti-inflammatory, anti-oxidant, and/or immuno-modulatory effect in the subject. As described herein, prolonged fasting induces improvements in the biochemical functionalities of human plasma, which are due to the increased plasma concentrations of several endogenous metabolites (e.g., spermidine, 1-MNA, PEA, and OEA or a precursor thereof) during fasting as identified by a comprehensive metabolic panel of subject plasma samples.

In some embodiments of the methods, the subject has an inflammatory disorder and would benefit from the anti-inflammatory effects provided by one or more of the metabolites spermidine, 1-MNA, PEA, and OEA or a precursor thereof. Examples of inflammatory disorders include, but are not limited to, rheumatoid arthritis (RA), systemic lupus erythematosus (SLE), ANCA-associated vasculitis, antiphospholipid antibody syndrome, autoimmune hemolytic anemia, chronic inflammatory demyelinating neuropathy, graft-vs-host disease (GVHD), dermatomyositis, Goodpasture's Syndrome, organ system-targeted type II hypersensitivity syndromes, Guillain Barre syndrome, chronic inflammatory demyelinating polyneuropathy (CIDP), dermatomyositis, Felty's syndrome, autoimmune thyroid disease, ulcerative colitis, autoimmune liver disease, idiopathic thrombocytopenia purpura, Myasthenia Gravis, neuromyelitis optica, pemphigus, Sjogren's Syndrome, autoimmune cytopenias, synovitis, dermatomyositis, systemic vasculitis, glomerulitis, and vasculitis. As demonstrated herein, prolonged fasting significantly increased the ability of subject plasma to suppress TNF-α secretion from primary macrophages that were stimulated with pro-inflammatory citrullinated-fibrinogen immune complexes. In some embodiments of the methods described herein, the amount of TNF-α secreted by macrophages after the subject receiving the one or more metabolites is less than 90% (e.g., less than 85%, less than 80%, less than 75%, less than 70%, less than 65%, less than 60%, less than 55%, less than 50%, less than 45%, less than 40%, less than 35%, less than 30%, less than 25%, less than 20%, less than 10%, or less than 5%) of the amount of TNF-α secreted by macrophages prior to the subject receiving the metabolite.

In some embodiments of the methods, the subject has one of the following disorders or suffers from inflammatory effects of the following disorders or diseases:
Viral infection or benefitting from protection from complications associated with pathogenic infections (viral, bacterial) (for example, but not limited to, sepsis, cytokine storm, systemic inflammatory response syndrome (SIRS), and severe acute respiratory syndrome (SARS)). Examples of viral infection include but are not limited to infection by severe acute respiratory syndrome coronavirus 2 (SARS-CoV-2), influenza, Marburg virus, Ebola, rabies, HIV, smallpox, hantavirus, Dengue virus, rotavirus, SARS-CoV, and MERS-CoV.
Neurodegenerative/neuroinflammatory disorders. Examples of neurodegenerative/neuroinflammatory disorders include but are not limited to Alzheimer's Disease (AD) and related dementias (e.g., ADRD), Parkinson's disease, Huntington's disease, frontotemporal dementia, progressive supranuclear palsy, corticobasalar degeneration, mild cognitive impairment, vascular dementia, Lewy body dementia, amyotropic lateral sclerosis, prion disorder, or HIV-related dementia.
Cardiovascular disease. Examples of Cardiovascular disease include but are not limited to coronary heart disease (CHD), coronary artery disease (CAD), acute myocardial infarction, myocardial ischemia, chronic heart failure, peripheral artery disease, critical limb ischemia, stroke (e.g., ischemic and hemorrhagic).
Diseases of aging. Diseases of aging include but are not limited to chronic inflammation (Ulcerative colitis, Crohns Disease, athersosclerosis, vascular disease, arthritis, diabetes, obesity, metabolic syndrome, chronically elevated clinical inflammatory makers), cognitive decline (dementia, Alzheimers disease, nuerodegenerative diseases), frailty, sarcopenia, and cancer.

Further, in some embodiments, the method can increase the total antioxidant capacity of the subject's plasma relative to the total antioxidant capacity of the subject's plasma prior to the subject receiving the metabolite. Total antioxidant capacity is a measure used to assess the antioxidant status of biological samples and can evaluate the antioxidant response against the free radicals produced in a given disease. Methods and techniques to measure total antioxidant capacity are available in the art, e.g., as described in Rubio et al., BMC Vet Res. 12:166, 2016 and Ialongo C. Review Clin Biochem 50(6):356-363, 2017. Commercially available tools and kits to measure total antioxidant capacity are also available, e.g., Cell Biolabs Catalog No. STA-360 and Abcam Catalog No. ab56329. In other embodiments, the methods can also decrease the amount of reactive oxygen species (ROS) produced by macrophages in the subject relative to the amount of ROS produced by macrophages in the subject prior to the subject receiving the metabolite. In some embodiments, the amount of ROS produced by macrophages in the subject after the subject received the one or more metabolites is less than 70% (e.g., less than 60%, less than 50%, less than 40%, less than 30%, less than 20%, or less than 10%) of the amount of ROS produced by macrophages in the subject prior to the subject received the one or more metabolites. Tools to measure the amount of ROS is available in the art, e.g., commercially available kit by Cell Biolabs Catalog No. STA-342).

In some embodiments of the methods, the subject has a metabolic disorder. For example, the metabolic disorder can include, for example, obesity, Type-1 diabetes, Type-2 diabetes, and atherosclerosis, non-alcoholic fatty liver disease (NAFLD), non-alcoholic steatohepatitis (NASH), and metabolic syndrome. Some symptoms of a metabolic disease include high serum triglycerides, high low-density cholesterol (LDL), low high-density cholesterol (HDL), and/or high fasting insulin levels, elevated fasting plasma glucose, abdominal (central) obesity, and elevated blood pressure.

In some embodiments, the methods can increase and promote the subject's cholesterol efflux, which refers to the transferring of intracellular cholesterol to extracellular acceptors, such as apolipoprotein A-I (apoA-I) of high-density lipoprotein (HDL). A variety of evidence shows that cholesterol efflux plays a major role in preventing atherosclerosis in humans (see, e.g., Phillips M., J Biol Chem. 289(35): 24020-24029, 2014). Methods and techniques to measure a subject's cholesterol efflux are available in the art, e.g., as described in Shimizu et al., J Lipid Res 60(11):1959-1967, 2019 and Norimatsu et al., Heart Vessels 32(1):30-38, 2017. Commercially available tools and kits to measure cholesterol efflux are also available, e.g., Abcam Catalog No. ab196985 and Sigma Catalog No. MAK192.

In some embodiments of the methods, the subject has a total cholesterol level of greater than 170 mg/dL (e.g., greater than 180 mg/dL, greater than 190 mg/dL, greater than 200 mg/dL, greater than 210 mg/dL, greater than 220 mg/dL, greater than 230 mg/dL, greater than 240 mg/dL, or greater than 250 mg/dL) and would benefit from being administered one or more of the metabolites spermidine, 1-MNA, PEA, and OEA. In certain embodiments, the subject has a low-density lipoprotein (LDL) level of greater than 100 mg/dL (e.g., greater than 110 mg/dL, greater than 120 mg/dL, greater than 130 mg/dL, greater than 140 mg/dL, greater than 150 mg/dL, greater than 160 mg/dL, or greater than 170 mg/dL). In certain embodiments, the subject has a high-density lipoprotein (HDL) level of less than 40 mg/dL for men or less than 50 mg/dL for women (e.g., less than 35 mg/dL, less than 30 mg/dL, less than 25 mg/dL, less than 20 mg/dL, less than 15 mg/dL, or less than 10 mg/dL).

In other embodiments, the subject is overweight. For example, the subject has a body mass index (BMI) of greater than 25 (or greater than 23 for Asian populations, or a waist circumference>35 inches for women and >40 inches for men) prior to administration of the one or more metabolites. In some embodiments, after receiving the one or more metabolites, the subject BMI is reduced to between 18 and 25 (e.g., between 18 and 24, between 18 and 23, between 18 and 22, between 18 and 21, between 18 and 20, between 18 and 19, between 19 and 25, between 20 and 25, between 21 and 25, between 22 and 25, between 23 and 25, between 24 and 25).

In further embodiments, the methods described herein extends longevity (e.g., causes lifespan extension, healthspan extension, healthy aging, alters biochemical pathways associated with the aging process or is used to treat or prevent age related diseases) and/or improves cognitive and/or physical performance of the subject. In some embodiments, the subject scores lower than 24 points on a Mini-Mental State Examination (MMSE) prior to administration of the one or more metabolites. In some embodiments, the subject scores 24 points or higher (e.g., between 24 and 30, between 24 and 29, between 24 and 28, between 24 and 27, between 24 and 26, between 24 and 25, between 25 and 30, between 26 and 30, between 27 and 30, between 28 and 30, or between 29 and 30) on the MMSE after administration of the one or more metabolites. The Mini-Mental State Examination (MMSE) is a 30-point questionnaire that is used extensively in clinical and research settings to measure cognitive impairment (Pangman et al., Applied Nursing Research. 13 (4):209-213, 2000). It is commonly used in medicine and allied health to screen for diseases with symptoms of decreased cognitive performance, e.g., dementia. It is also used to estimate the severity and progression of cognitive impairment and to follow the course of cognitive changes in an individual over time; thus making it an effective way to document an individual's response to treatment. In some embodiments, a score of 24 or more (out of 30) indicates a normal cognition. Below this, scores can indicate severe (≤9 points), moderate (10-18 points), or mild (19-23 points) cognitive impairment. In some embodiments, the subject has a MMSE score of 9 points or less (e.g., 8, 7, 6, 5, 4, 3, 2, or 1 point) before receiving the metabolites. In some embodiments, the subject has a MMSE score of 10 to 18 points (e.g., 10, 11, 12, 13, 14, 15, 16, 17, or 18 points) before receiving the metabolites. In some embodiments, the subject has a MMSE score of 19 to 23 points (e.g., 19, 20, 21, 22, or 23 points) before receiving the metabolites.

Canonically, there are 9 known hallmarks of aging: 1. Altered intracellular communication 2. Stem cell exhaustion 3. Mitchondiral dysfunction 4. Cellular senescence 5. Deregulated nutrient sending 6. Loss of proteostasis 7. Epigeneitic alterations 8. Telomere attrition 9. Genomic instability. The molecular pathways and processes that are involved with these hallmarks of aging would be what we're talking about in terms of “pathways associated with the aging process or prevention of age related diseases” those pathways would also include mTOR, APMK, MAPK, JAK/STAT, NAD/NADH, SIRT, FOXO, autophagy, mitophagy, telomerase activity, mitochondrial functionality ROS generation, IGF-1, p53, AGEs. In some embodiments, the compositions described herein improve one or more of these hallmarks of aging.

In some embodiments of the methods, the subject is on a fasting diet. A fasting diet refers to a diet that has at least 5 hours (e.g., at least 6, 8, 10, 12, 14, 16, 18, 20, 22, 24, 26, 28, 30, 32, 34, 36, 38, 40, or 42 hours) in between consumption of any food. In some embodiments, the one or more metabolites are administered to the subject one or more times daily (e.g., once, twice, three times, four times, or five times). In some embodiments, the metabolite is administered to the subject during food intake. In other embodiments, the metabolite is administered to the subject after at least 5 hours (e.g., at least 6, 8, 10, 12, 14, 16, 18, 20, 22, 24, 26, 28, 30, 32, 34, 36, 38, 40, or 42 hours) of fasting. In certain embodiments, when the subject is on a fasting diet (e.g., fasting for more than 12 hours (e.g., between 12 and 15 hours, between 15 and 20 hours, between 20 and 25 hours, between 25 and 30 hours, between 30 and 36 hours, or more than 36 hours)), one or more of metabolies selected from the group consisting of pentose acid, indolepropionate, gentisate, piperine, and hydrocinnamate are substantially depleted in the subject.

Further, in some embodiments, the methods can decrease cyclooxygenase (COX) activity and relieve pain in the subject. As demonstrated, treating macrophages with plasma isolated from subjects who were on a fasting diet induced a decrease in the total COX activity of the macrophages, which indicated a role of COX signaling in immunomodulation. Moreover, the methods described herein can also decrease nitric oxide synthases (NOS) activity in the subject. A decrease in NOS activity is associated with M1 polarization of macrophages, which is often referred to as the pro-inflammatory type of macrophages that are important in the defense against pathogens and the secretion of pro-inflammatory cytokines. Further, the methods described herein can also increase arginase activity in the subject. An increase in ariginase activity is associated with M2 polarization of macrophages, which is often involved in regulation of inflammation and repair of damaged tissues.

The inventors have also found that the combined four metabolites described herein increase plasma cholesterol efflux ability in human subjects: Increasing plasma efflux ability is a measure of the cardioprotective ability of the plasma and standard clinical marker of cardiovascular disease risk. Accordingly, the disclosure provides methods of preventing or treating heart disease, stroke, arteral plaque formation or other cardiovascular disease risk factors in a subject by administering the four metabolites described herein or a precursor thereof. Exemplary subjects that can benefit from this effect are at risk for or have heart disease, stroke, arteral plaque formation or other cardiovascular disease risk factor.

### V. Pharmaceutical Compositions and Routes of Administration

The disclosure features pharmaceutical compositions that include one or more of the metabolites spermidine, PEA, OEA, and 1-MNA and one or more pharmaceutically acceptable carriers or excipients, which can be formulated by methods known to those skilled in the art.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. Pharmaceutical compositions of the disclosure can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2nd ed.) Taylor & Francis Group, CRC Press (2006).

The pharmaceutical compositions of the disclosure may be prepared in various forms depending on the mode of administration. In some embodiments, the pharmaceutical compositions of the disclosure can be prepared in microcapsules, such as hydroxylmethylcellulose or gelatin-microcapsule and poly-(methylmethacrylate) microcapsule. The pharmaceutical composition can be formed in a unit dose form as needed. The amount of active component, e.g., one or more of the metabolites spermidine, PEA, OEA, and 1-MNA or a precursor thereof, included in the pharmaceutical preparations is such that a suitable dose within the designated range is provided.

Pharmaceutical compositions that contain one or more of the metabolites can be formulated for various routes of administration, such as oral administration, intravenous administration, parenteral administration, transdermal, topical or intraperitoneal administration. In particular, the pharmaceutical composition is formulated for oral administration. For example, the pharmaceutical composition can be formulated for oral administration as one or more pills, one or more tablets, or one or more bottles of syrup or powder than can be mixed into different foods and beverages. In some embodiments, the pharmaceutical composition is administered to the subject one or more times daily (e.g., once, twice, three times, four times, or five times). In certain embodiments, the pharmaceutical composition can be administered to a subject who is on a fasting diet (e.g., a prolonged fasting diet). The pharmaceutical composition can be administered to the subject during food intake. In other embodiments, the pharmaceutical composition can be administered to the subject during a time that is in between food consumptions. In some embodiments, the pharmaceutical composition can be administered to the subject after food intake, e.g., at least 1 hour (e.g., at least 1, 2, 3, 4, 5, 6, 7, 8, 9, or 10 hours) after food intake.

The dosage of the pharmaceutical compositions depends on factors including the route of administration, the disease to be treated, and physical characteristics, e.g., age, weight, general health, of the subject. Typically, the amount(s) of the one or more metabolites in the pharmaceutical composition within a single dose may be an amount that effectively incudes an anti-inflammatory, anti-oxidant, and/or immuno-modulatory effect in the subject without inducing significant toxicity. A pharmaceutical composition of the invention may include a dosage of a metabolite ranging from 0.01 to 500 mg/kg (e.g., 0.01 to 500, 0.01 to 400, 0.01 to 300, 0.01 to 200, 0.01 to 100, 0.01 to 90, 0.01 to 80, 0.01 to 70, 0.01 to 60, 0.01 to 50, 0.01 to 40, 0.01 to 30, 0.01 to 20, 0.01 to 10, 0.01 to 1, 0.1 to 500, 1 to 500, 10 to 500, 20 to 500, 30 to 500, 40 to 500, 50 to 500, 60 to 500, 70 to 500, 80 to 500, 90 to 500, 100 to 500, 200 to 500, 300 to 500, or 400 to 500 mg/kg). The dosage may be adapted by the physician in accordance with conventional factors such as the extent of the disease and different parameters of the subject. In some embodiments, pharmaceutical compositions that contain one or more of the metabolites spermidine, PEA, OEA, and 1-MNA can be administered to a subject in need thereof, for example, one or more times (e.g., 1, 2, 3, 4, 5, 6, 7, 8, 9, or 10 times or more) daily, weekly, monthly, biannually, or annually. Dosages may be provided in either a single or multiple dosage regimens. The timing between administrations can decrease as the medical condition improves or increase as the health of the patient declines.

### EXAMPLES

The following examples are intended to illustrate, but not to limit the present disclosure.

### Example 1. Study Design and Methods

Human Clinical Trial of PF

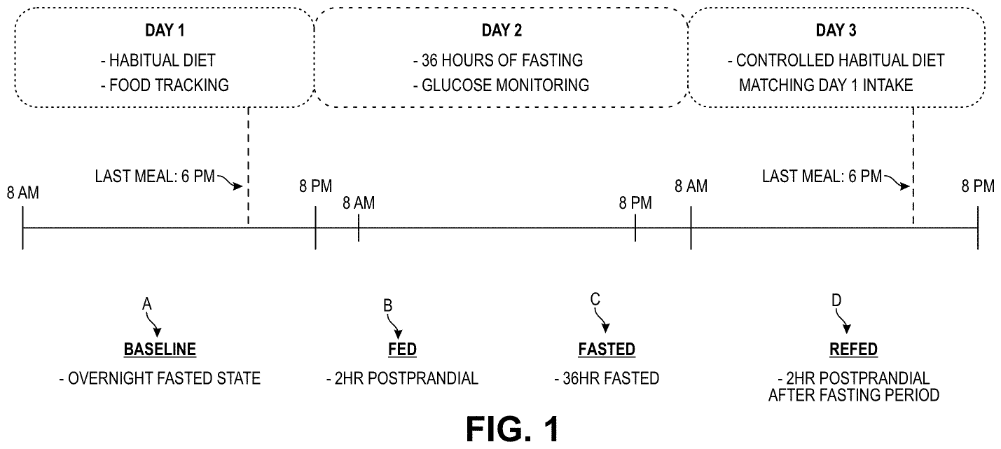

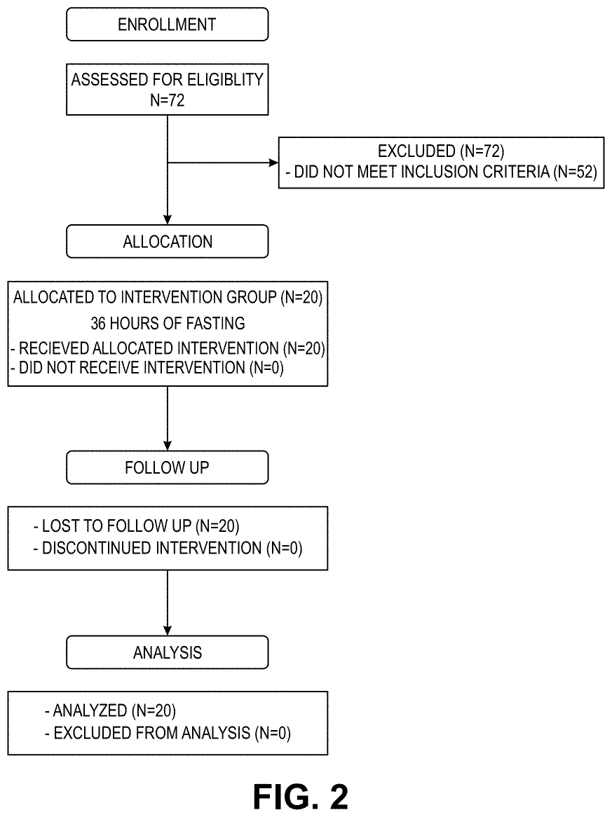

In order to assess the effects of prolonged fasting (PF) on the plasma metabolome and macrophage functionalities of human participants, a 3-day human study of a single bout of 36 hours of fasting in 20 young healthy participants (FIG. 1) was performed. Full clinical study protocols can be found under ClinicalTrials ID NCT03487679. Briefly, participants were included if they were 20-40 years old, had a BMI within the range 19-27 kg/m2, had fasting glucose in the clinically normal range of 70-100 mg/dL, had no documented health conditions, and no extreme dietary or exercise patterns. Seventy-two interested people were screened for eligibility and 20 were found to meet inclusion and exclusion criteria and enrolled in the study (FIG. 2). There were no drop outs, adverse events, or protocol violations, and all 20 participants successfully completed the 3-day trial and were included in experimental analysis (FIG. 2). All clinical study activities were performed at the UC Davis Ragle Human Nutrition Research Center.

On Day 1, participants in an overnight (12 hr) fasted state provided a baseline blood draw at approximately 8 AM representing the Baseline state and were then instructed to go about their normal routine and importantly, were instructed to consume their habitual diet while keeping track of their dietary intake (Table 1) using a detailed food record. Participants were instructed to eat their last meal at 6 PM on Day 1 and then returned to the Ragle Center for a 2 hr postprandial blood draw at 8 PM representing the Fed state. Participants then underwent a period of 36 hrs of fasting representing the rest of Day 1 and all of Day 2 during which they were monitored for compliance utilizing glucose meters. On Day 3 at approximately 8 AM participants provided a 36 hr fasted blood draw representing the Fasted state and were then given a copy of their recorded dietary intake from Day 1. Participants were then instructed to eat exactly the same diet as they recorded on Day 1 throughout all of Day 3 and, as before, ate their last meal at 6 PM and received a final 2 hr postprandial blood draw representing the Refed state. In Table 1 below, baseline values were determined from 24 hr food recalls of the previous day during the Baseline visit. Fed values were determined from food intake recorded throughout Day 1 of the study. Refed values were determined from food intake recorded throughout Day 3 of the study. There were no significant differences observed between any state throughout the course of the study.

TABLE 1
Nutrient Intake of Study Participants

| | Baseline | Fed | Refed | pval_ba | pval_da | pval_db |
|---|---|---|---|---|---|---|
| Cals (kcal) | 2020 ± 673.8 | 1902 ± 564.2 | 1964 ± 553.2 | 0.648 | 0.648 | 0.648 |
| FatCals (kcal) | 741 ± 403 | 683.2 ± 307.9 | 710.7 ± 331.3 | 0.66 | 0.66 | 0.66 |
| SatCals (kcal) | 234.4 ± 178.4 | 182.1 ± 104.9 | 184.4 ± 102.1 | 0.111 | 0.111 | 0.933 |
| Prot (g) | 88.6 ± 43.1 | 79.1 ± 34.3 | 80.5 ± 33.9 | 0.344 | 0.344 | 0.833 |
| Carb (g) | 233 ± 83.6 | 231.5 ± 70.2 | 239.8 ± 68.7 | 0.921 | 0.921 | 0.921 |
| Fib (g) | 27.6 ± 11.7 | 32.3 ± 13.9 | 32.6 ± 13.9 | 0.149 | 0.149 | 0.924 |
| SolFib (g) | 2.3 ± 3.7 | 2.6 ± 2.7 | 2.5 ± 2.8 | 0.837 | 0.837 | 0.837 |
| Sugar (g) | 73.7 ± 40.7 | 70.4 ± 25.7 | 76.3 ± 27.8 | 0.746 | 0.746 | 0.746 |
| MonSac (g) | 13.6 ± 12.5 | 12.5 ± 13.7 | 13.1 ± 14.6 | 0.841 | 0.841 | 0.841 |
| Disacc (g) | 10.3 ± 16.3 | 5.6 ± 5.2 | 5.8 ± 5.6 | 0.215 | 0.215 | 0.955 |
| OCarb (g) | 112.8 ± 63.6 | 110.5 ± 39.8 | 112.7 ± 41.6 | 0.997 | 0.997 | 0.997 |
| Fat (g) | 82.5 ± 45 | 76.1 ± 34.4 | 79.2 ± 37 | 0.66 | 0.66 | 0.66 |
| SatFat (g) | 26 ± 19.8 | 20.2 ± 11.7 | 20.5 ± 11.3 | 0.111 | 0.111 | 0.932 |
| MonoFat (g) | 19.2 ± 17.9 | 18.7 ± 16.2 | 19.7 ± 17.5 | 0.88 | 0.88 | 0.88 |
| Poly Fat (g) | 8.2 ± 4.5 | 9 ± 9.1 | 9.5 ± 9.1 | 0.718 | 0.718 | 0.718 |
| TransFat (g) | 0.6 ± 0.8 | 0.2 ± 0.3 | 0.2 ± 0.4 | 0.053 | 0.053 | 0.862 |
| Chol (mg) | 289 ± 247.2 | 235.3 ± 158.2 | 229.7 ± 172.1 | 0.244 | 0.244 | 0.883 |
| Water (g) | 2528 ± 1169 | 2491 ± 1674 | 2595 ± 1667 | 0.906 | 0.906 | 0.906 |

This methodology is termed a “controlled habitual diet” in monitoring and controlling food intake during a clinical study. A controlled habitual diet as implemented here offers several advantages to nutritional studies, particularly cross-over studies where each participant acts as their own control. Importantly, it allows researchers to control for nutritional intake between states while avoiding the well-known disruptive metabolic effects of introducing a novel standardized diet into disparate human populations²⁴. Unlike other fasting trials, utilizing a controlled habitual diet as well as assessing 4 distinct nutritional states from each individual throughout the course of the study allowed for a clear comparison of not only the postprandial state to the 36 hr fasted state, but also the assessment of the distinct effects of PF beyond a typical overnight fast and the potential carry over effects of PF to a refed postprandial state after fasting.

Blood Processing

All participant blood samples were collected in EDTA plasma tubes and immediately processed to yield participant plasma. Participant plasma was immediately stored at −80° C.

NMR Lipoprofile

Plasma lipoprotein particle sizes and concentrations were analyzed by proton nuclear magnetic resonance (NMR) spectroscopy at LabCorp (LipoScience, Inc., Morrisville, NC). This analysis, certified according to Clinical and Laboratory Standards Institute (CLSI) EP5-A2 guidelines, uses NMR to estimate the number and size of lipoprotein particles within the lipoprotein subclasses including VLDL, LDL, and HDL particles from small to large, reporting a calculated Lipoprotein Insulin Resistance (LP-IR) index²⁵, which has been shown to be highly correlated with multiple indices of insulin resistance and predictive of incident type 2 diabetes across large multi-ethnic cohort studies²⁵⁻²⁷. Included in the report are total cholesterol, LDL-C, HDL-C, and triglycerides calculated from the NMR spectral data. An additional result from this test panel includes GlycA, a measurement of NMR signal from acute phase proteins, which measures the overall degree of inflammation in plasma and which has been shown to be associated with incident cardiovascular disease, cardiometabolic risk, and a number of inflammatory conditions including rheumatoid arthritis^{28,29}. Finally, the test panel also provides concentrations of ketone bodies, glucose, and total protein.

Metabolomic Analyses

Metabolomic analysis was conducted at Metabolon Inc. (Morrisville, North Carolina) as previously described³⁰. Briefly, samples were homogenized and subjected to methanol extraction then split into aliquots for analysis by ultrahigh performance liquid chromatography/mass spectrometry (UHPLC/MS) in the positive (two methods) and negative (two methods) mode. Metabolites were then identified by automated comparison of ion features to a reference library of chemical standards followed by visual inspection for quality control as previously described³¹. For statistical analyses and data display, any missing values are assumed to be below the limits of detection; these values were imputed with the compound minimum (minimum value imputation).

Primary Macrophage Isolation

Primary macrophage for use in experimental in vitro assays of participant plasma functionalities were isolated from healthy volunteers in an overnight fasted state. PBMCs were isolated using Ficoll gradient extraction, then cultured in flasks with Rosewell Park Memorial Institute Medium 1640 (RMPI) (Thermo Fisher, 11875119), 1× Penicillin-Streptomycin-Glutamine (PSG) (Thermo Fisher, 11875119) for 3 hrs to induce adhesion. Nonadherent cells were discarded and adherent cells were placed into RPMI, 10% Fetal Bovine Serum (FBS) (Thermo Fisher, A3160402), 1×PSG containing 20 ng/mL human macrophage colony stimulating factor for 7 days to induce macrophage differentiation.

THP-1 Macrophage Differentiation

For certain assays requiring high cellular concentrations beyond what was feasible to isolate from human volunteers, THP-1 monocytes (ATCC, TIB-202) were cultured in RPMI 1640, 10% FBS, lx PSG and differentiated into M0 macrophage using 100 nM phorbol 12-myristate-13-acetate (PMA) for 2 days followed by incubation with clean RPMI, 10% FBS, 1×PSG for 1 day.

### Example 2. Functional Analyses of Participant Plasma and Isolated Metabolites

Analysis of molecular functionalities of participant plasma from each time point and in vitro effects of participant plasma and isolated metabolites on human macrophage functionalities and activities were performed as described below.

Citrullinated Fibrinogen Immune Complex Assay

The responsiveness of treated primary macrophage to citrullinated fibrinogen immune complexes (cFb ICs), an FC-γ receptor stimulant and in vitro model of autoimmune disease³², was performed as previously described³². Briefly, 20 μg/mL of citrullinated fibrinogen was plated onto 96-well plates, blocked, and treated with 20 μg/mL of anti-fibrinogen antibody (Agilent, A008002-2) to form cFb ICs. Primary human macrophage in RPMI 1640, 1×PSG were treated with participant plasma to a final concentration of 10%, isolated metabolites, or FBS to a final concentration of 10% as a positive control for 1 hr and then plated at 5.0×10⁵ cells/mL and allowed to incubate at 37° C. overnight (approximately 18 hrs). A negative control well containing macrophage in RPMI 1640, 10% FBS, 1×PSG without immune complexes was also included. TNF-α secretion from treated macrophage was then measured in the cell supernatant via ELISA (PeproTech, 900-K25). After obtaining the results from the initial experiment of participant plasma on macrophage reactivity in this model using a single primary cell donor, a follow up experiment was performed to confirm the observed results in a second primary cell donor. From those results, it was found the same significant effects of participant plasma on macrophage reactivity from both primary cell donors and so proceeded to use macrophage from a single consistent cell donor for the remainder of the analyses utilizing primary macrophage. (Inter CV: 7.8, Intra CV: 10.3)

Antioxidant Capacity

Plasma antioxidant capacity was assessed using a commercially available kit (Abcam, ab65329) (Inter CV: 6.7, Intra CV: 9.45)

Cholesterol Efflux Ability

Cholesterol efflux ability of participant plasma from primary macrophage was measured using a commercially available kit (Abcam, ab196985) with the following modifications: Primary macrophage were lipid loaded with one half of the recommended labeled cholesterol for 4 hours prior to incubation with either participant plasma in equilibration buffer to a final concentration of 1% or isolated metabolites in equilibration buffer. Cholesterol efflux from macrophage was measured after 2 hours of incubation. (Inter CV: 7.3, Intra CV: 9.8)

Intracellular ROS Assay

The effect of treatment with participant plasma at a final concentration of 20% and individual metabolites on cellular reactive oxygen species (ROS) production in primary macrophage was assessed using a commercially available kit (Cell Biolabs, STA-342). (Inter CV:6.7, Intra CV: 11.2)

COX Activity Assay

The effect of treatment with participant plasma and individual metabolites on the total cellular COX activity of THP-1 macrophage was measured using a commercially available kit (Cayman Chemical, 760151). In order to induce COX-2 expression, THP-1 macrophage were incubated with 10 ng/mL of lipopolysaccharide (LPS) (Sigma, LPS25) in RPMI, 1×PSG along with 20% participant plasma, or in RMPI, 10% FBS, 1×PSG either alone as a positive control or with isolated metabolites overnight (approximately 18 hrs) before measuring total COX activity from cell lysates. A negative control of macrophage in RPMI, 10% FBS, 1×PSG without LPS was also performed. (Inter CV: 5.4, Intra CV: 12.3)

M1 Polarization Assay

The effect of treatment with participant plasma and individual metabolites on the induction of M1 polarization was assessed via enzymatic analysis of nitric oxide synthase (NOS) and arginase activity in macrophage cell lysates. M0 THP-1 macrophage were incubated for 2 days with 100 ng/mL LPS and 20 ng/mL interferon gamma, a known M1 polarization inducer³³, in either RPMI 1640, 1×PSG with 20% participant plasma or RPMI, 10% FBS, 1×PSG alone as a positive control or with isolated metabolites. A negative control of macrophage in RPMI, 10%, 1×PSG without LPS and INF-γ was also performed. NOS and arginase activities of these cells were assessed in cell lysates standardized to total protein content via commercially available kits (Abcam, ab211083, ab180877). ROS (Inter CV: 8.7, Intra CV: 10.6) Arginase (Inter CV: 9.2, Intra CV: 11.0)

### Example 3. C. Elegans Lifespan Studies

C. elegans var. Bristol (N2), was used as the wild-type strain. Strains were maintained and lifespan assays were performed at 20° C. Twenty-four hours after seeding Escherichia coli (OP50) bacteria on standard NGM plates, the bacteria were killed by 4 min exposure to UV irradiation using a Stratalinker UV crosslinker (Stratagene, Model 2400).

The following concentrations of compounds were used: 0.2 mM spermidine, 0.5 mM 1-MNA, 0.01 and 0.1 mM PEA, and 0.01 mM OEA. Spermidine and MNA were diluted into 100 μl sterilized water and applied to the top of the agar medium (3 ml NGM plates). Plates were then gently swirled to allow compounds to spread to the entire NGM surface. Identical solutions of compound-free water were used for the control plates. Plates were then allowed to dry overnight. The procedure was repeated each time worms were transferred to fresh plates (every 2-4 days). PEA and OEA were added to cooled agar, prior to solidification; for the combination, all compounds were added to cooled agar and plates stored at 4° C. Synchronous worm populations were generated by hypochlorite treatment of gravid adults and life span assays were performed beginning at the L4 stage (Day 0). Seven to eight plates of 15 worms each were exposed to the indicated compounds. Animals were transferred to fresh plates every 2 days for the first week and then 3-4 days thereafter. Worms were examined for touch-provoked movement and pharyngeal pumping, until death. Worms that died due to desiccation from crawling on the edge of the plates, were censored.

### Example 4. Statistical Analyses

Metabolomic Analyses

All statistical analyses were performed in the statistical language R (3.6.1). A linear model was used to test the differences between experimental groups by giving each subject an individual intercept using the R package limma³⁴; P<0.05 is considered significant. Multiple comparisons were corrected using the Benjamini-Hochberg method. The MS intensity of each metabolite was log transformed prior to group comparisons. Fisher's exact test was performed to test whether metabolites that were significantly increased or decreased (p<0.05) were enriched in particular pathways using Metabolon's Portal Database.

In Vitro Functional Assessments

An ANOVA mixed model was fit for each in vitro functional assay using the treatment group as a fixed variable and subject ID as a random variable. The ANOVA model was fit using the R package lme4 (1.1.21)³⁵. Post-hoc comparison was performed on the fitted ANOVA model using Tukey's all-pair comparisons with the R package multcomp (1.4.13)³⁶.

C. elegans Lifespan Analysis

Kaplan-Meier survival curves were created to evaluate C. elegans lifespan. The R package survival (3.1.8) was used to fit the Cox proportional hazards regression model to evaluate differences of survivals between each treatment group versus control.

### Example 5. Human Trial of Prolonged Fasting

In order to assess the effects of PF on the metabolome and macrophage functionalities of human participants we performed a 3-day human study of 36 hours of fasting in 20 young healthy participants (Age: 27.5 yrs±4.35, BMI: 24.1 kg/m2±2.66, Men: n=10, Women: n=10). Across the trial, plasma was collected during 4 distinct nutritional states including an overnight fasted baseline state (Baseline), a 2 hr postprandial state (Fed), a 36 hr fasted state (Fasted), and a second 2 hr postprandial state after 36 hrs of fasting (Refed). A timeline of the study protocol can be found in FIG. 1 and baseline characteristics of participants can be found in Table 2.

TABLE 2

| | Baseline value |
|---|---|
| Age (yrs) | 27.5 ± 4.35 |
| Height (cm) | 170.7 ± 9.8 |
| Weight (kg) | 71.3 ± 13.4 |
| BMI | 24.3 ± 3.1 |
| Waist Circumference (cm) | 78.8 ± 8.9 |
| Systolic Blood Pressure (mmHg) | 113.7 ± 9 |
| Diastolic Blood Pressure (mmHg) | 71.6 ± 5.5 |

Of 20 participants enrolled, all successfully completed the study protocol without any protocol violations and were included in experimental analyses (FIG. 2). Compliance to fasting was assessed through the use of personal glucose monitoring throughout the waking hours of the fasting period as well as assessment of elevated ketone bodies in the Fasted state (Table 3). All participants were found to be compliant to the fasting period as indicated by elevated ketone bodies in the Fasted state above the values of the Baseline state (Table 3) and consistent glucose readings below 100 mg/dL throughout the fasting period. Typical of fasting, nuclear magnetic resonance (NMR) lipoprofile data from each timepoint showed significantly increased circulating ketone bodies and amino acids along with significantly decreased glucose values in the Fasted versus the Baseline state (Table 3). Similarly, circulating triglycerides were significantly reduced and ketone bodies and glucose levels significantly elevated in the Refed State versus the Fed state indicating that even after a full day of eating there are still metabolic carryover effects of PF (Table 3). Interestingly, LDL cholesterol levels were also significantly elevated in the Fasted state versus the Baseline state while HDL cholesterol levels were unchanged indicating potentially altered lipoprotein and cholesterol metabolism in response to PF (Table 3). Table 3 shows average NMR lipoprofile data from 20 study participants across timepoints. Significance values are given comparing Baseline state (A) to Fasted state (C) and Fed state (B) to Refed state (D). Elevated ketone bodies in the Fasted versus the Baseline indicate compliance to fasting.

TABLE 3
NMR Liproprofile Data of Study Participants

| | A | B | C | D | pval_ac | pval_bd |
|---|---|---|---|---|---|---|
| HDL-C (mg/dL) | 66.1 ± 16.8 | 68.2 ± 17.5 | 67.7 ± 15.1 | 67.5 ± 15.9 | 0.622 | 0.948 |
| LDL-C (mg/dL) | 77.5 ± 27 | 76.8 ± 29.4 | 85.8 ± 25.1 | 82.2 ± 30.1 | <0.001 | 0.058 |
| Triglyceride | 94.4 ± 31.6 | 118.6 ± 49.3 | 81.8 ± 17.8 | 84 ± 28 | 0.091 | <0.001 |
| (mg/dL) | | | | | | |
| Total Cholesterol | 163.1 ± 36.3 | 168 ± 39.2 | 166.8 ± 29.2 | 165.2 ± 37 | 0.417 | 0.594 |
| (mg/dL) | | | | | | |
| Glucose (mg/dL) | 86.4 ± 11.4 | 88.4 ± 14.1 | 74.5 ± 9.1 | 97.1 ± 10.3 | <0.001 | 0.014 |
| Ketone Bodies | 191.6 ± 129.9 | 187.1 ± 160.6 | 2284 ± 1261 | 353.1 ± 334.4 | <0.001 | <0.001 |
| (umol/L) | | | | | | |
| Protein (a.u.) | 530.2 ± 52.8 | 537.5 ± 47.7 | 561.1 ± 33.2 | 550.9 ± 43 | <0.001 | 0.129 |
| LPIR^{†} | 19.8 ± 13.5 | 22.9 ± 13.1 | 12.7 ± 5.6 | 16.4 ± 8.7 | 0.166 | 0.161 |
| GlycA (umol/L) | 329.9 ± 54.9 | 325 ± 48.6 | 316.4 ± 43.9 | 317.6 ± 50 | 0.154 | 0.318 |

Values presented as mean ± standard deviation.
Lipoprotein Insulin Resistance Index score from 0-100.

### Example 6. Pf Enhances Plasma Functionalities and Induces Anti-Inflammatory Effects in Macrophage

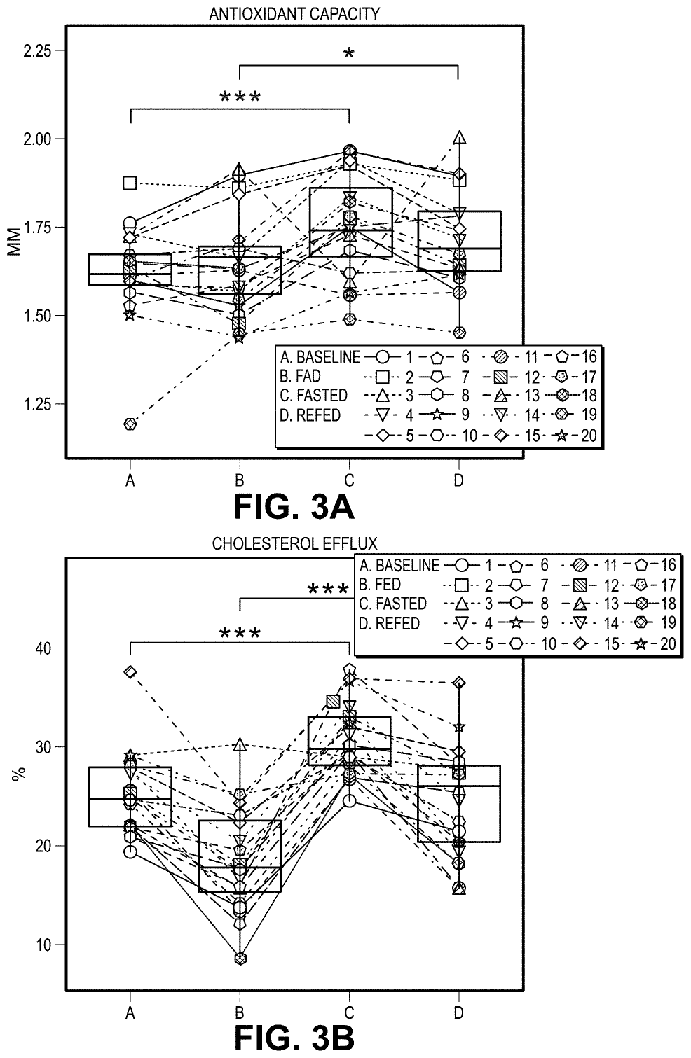

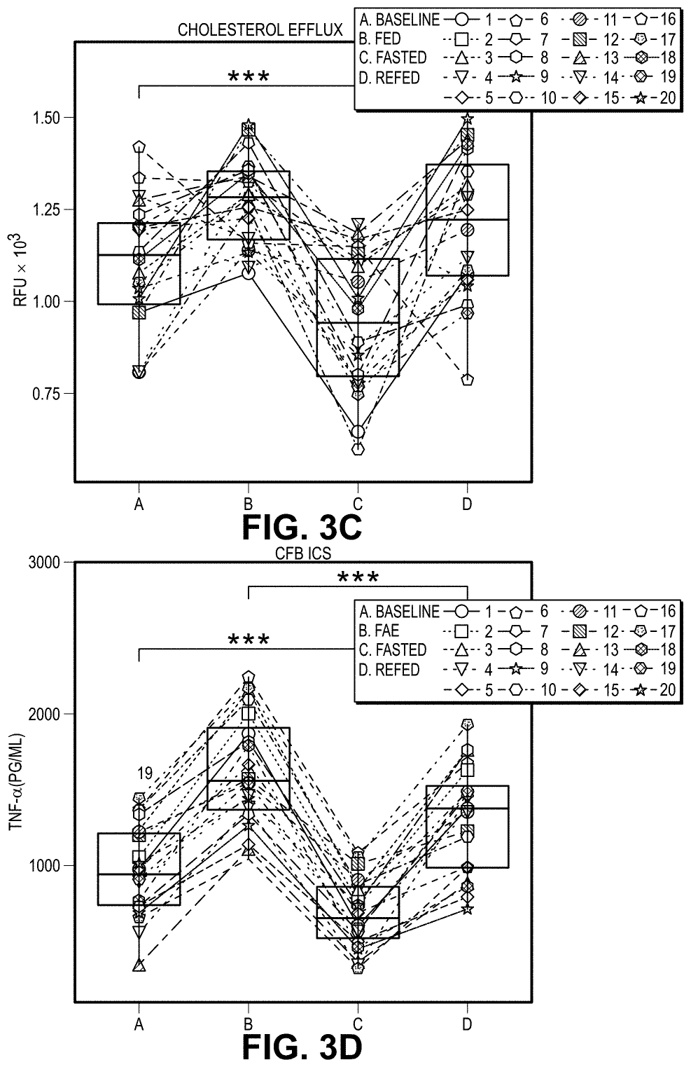

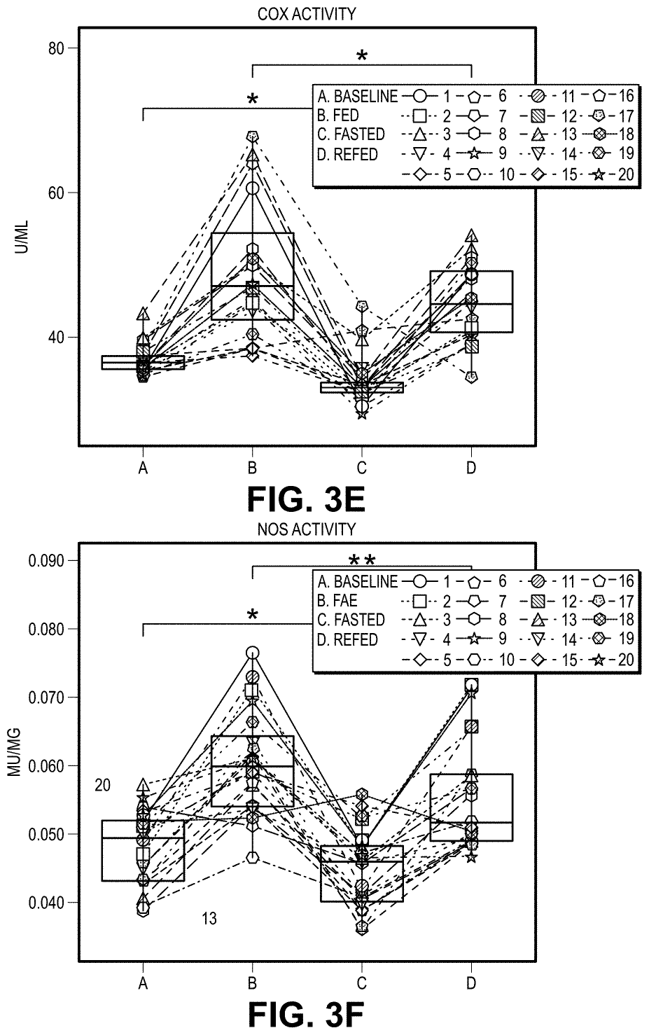

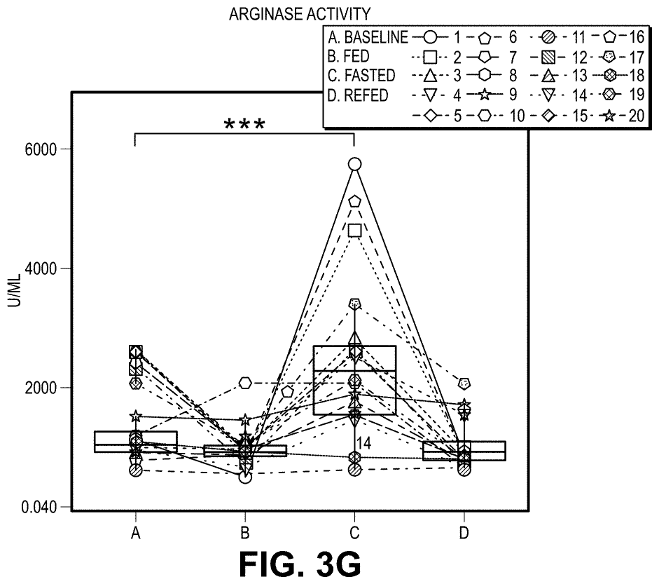

In order to assess the molecular effects of PF on participant plasma, multiple biochemical assessments of the functionality of participant plasma across states as well as the effect of exposure to participant plasma on the functionalities of human macrophage were performed. For all assessments reported, significant differences were found between not only the Fed and Fasted states, but also the Baseline and Fasted states indicating unique effects of PF that are not achieved through an ordinary overnight fast. Firstly, it was found that PF was capable of significantly increasing the total antioxidant capacity of human plasma from both the Fed and Baseline states and strikingly that that this effect was also achieved in the Refed state indicating a powerful carry-over effect of PF to the refed postprandial state even after a full day of habitual eating (FIG. 3A). Similarly, it was found that treatment of primary human macrophages with participant plasma was capable of significantly decreasing cellular reactive oxygen species (ROS) production in the Fasted state versus the Baseline and Fed states (FIG. 3C). It was also found that the activity of participant plasma to efflux cholesterol from lipid-loaded macrophage in the Fasted state was also significantly increased from the Baseline and Fed states and that the Refed state had significantly higher efflux ability than the Fed state (FIG. 3B).

The effects of participant plasma on primary human macrophage was also assessed in a previously described in vitro model of autoimmune disease³². Remarkably, a highly significant decrease in pro-inflammatory TNF-α secretion from primary human macrophage treated with Fasted plasma vs Baseline and Fed plasma during stimulation with cFb ICs was found and this effect carried over to the Refed state compared to the Fed state (FIG. 3D). To further investigate the scope of the observed immunomodulation, the effects of treatment of THP-1 macrophage with participant plasma during in vitro induction of M1 polarization³³ were assessed. It was found that treatment with participant plasma from the Fasted state versus the Fed and Baseline states significantly decreased cellular NOS activity whose activity is highly associated with classical activation and M1 polarization³³ along with concomitant increases in arginase activity, whose activity is highly associated with alternative activation and M2 polarization³³, in Fasted plasma treated versus Baseline and Fed plasma treated cells (FIGS. 3F and 3G).

These results indicate, for the first time in humans, that treatment with Fasted plasma is capable of affecting the polarization state of macrophage. Furthermore, that Fasted plasma is capable of modulating cells away from classical activation responses and towards alternative activation responses. In order to further elucidate the cellular pathways that may be involved in these immunomodulatory effects, the effects of treatment with participant plasma on the total COX activity of THP-1 macrophage during LPS stimulation were assessed. It was found that treatment with Fasted plasma was able to significantly decrease total COX activity versus the Baseline and Fed plasma treated cells, indicating a previously unknown role of COX signaling in PF induced immunomodulation (FIG. 3E). These results show, for the first time, PF significantly alters the functionality of human plasma, that the anti-inflammatory effects of PF are inducible even in non-fasted macrophage through treatment with participant plasma in a 36 hr fasted state and, furthermore, that these effects are quantitatively larger than a typical overnight fast. These results suggested that there may be numerous plasma-borne factors that are differentially regulated between the Baseline and Fasted states that mediate the enhanced functional effects observed in the Fasted plasma. In order to identify these factors, comprehensive metabolomics on each participant plasma sample across all 4 nutritional states were performed.

### Example 7. Pf Dramatically and Uniquely Alters the Human Plasma Metabolome

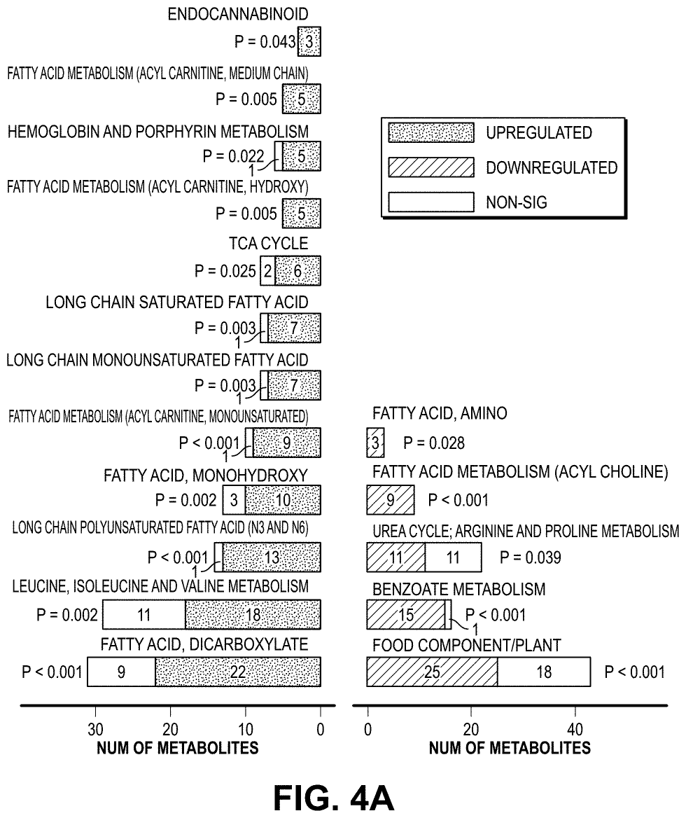

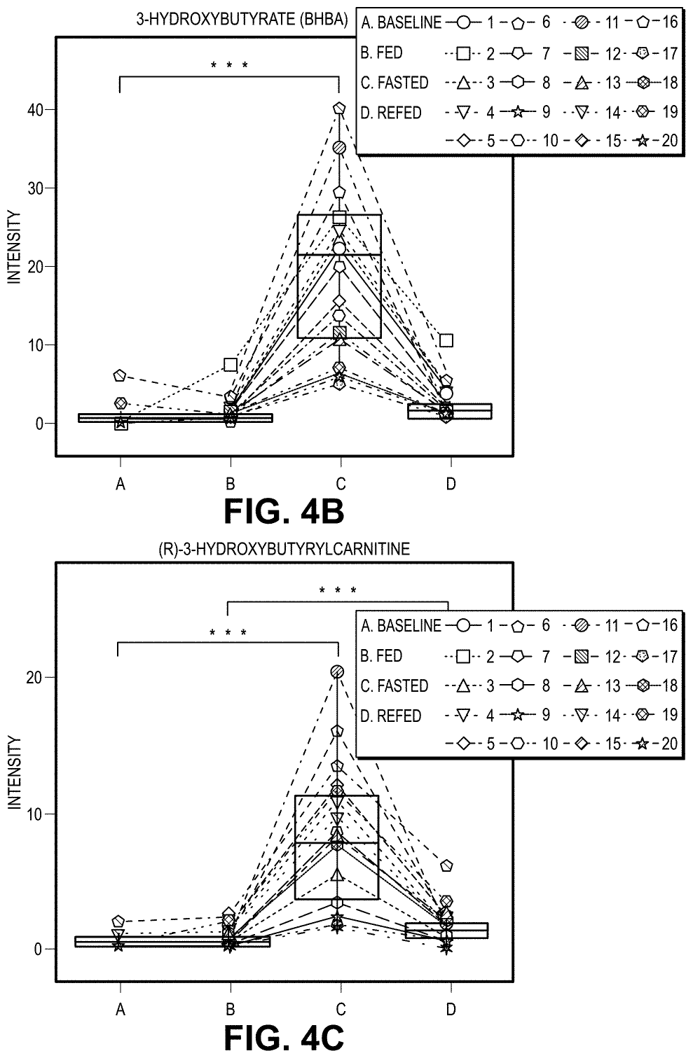

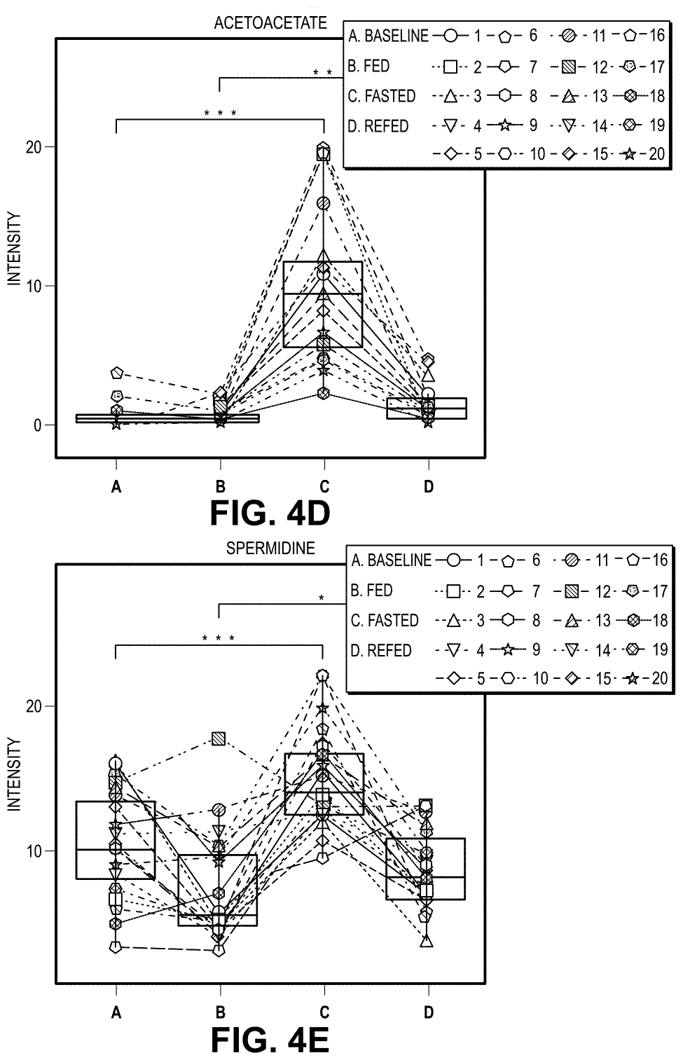

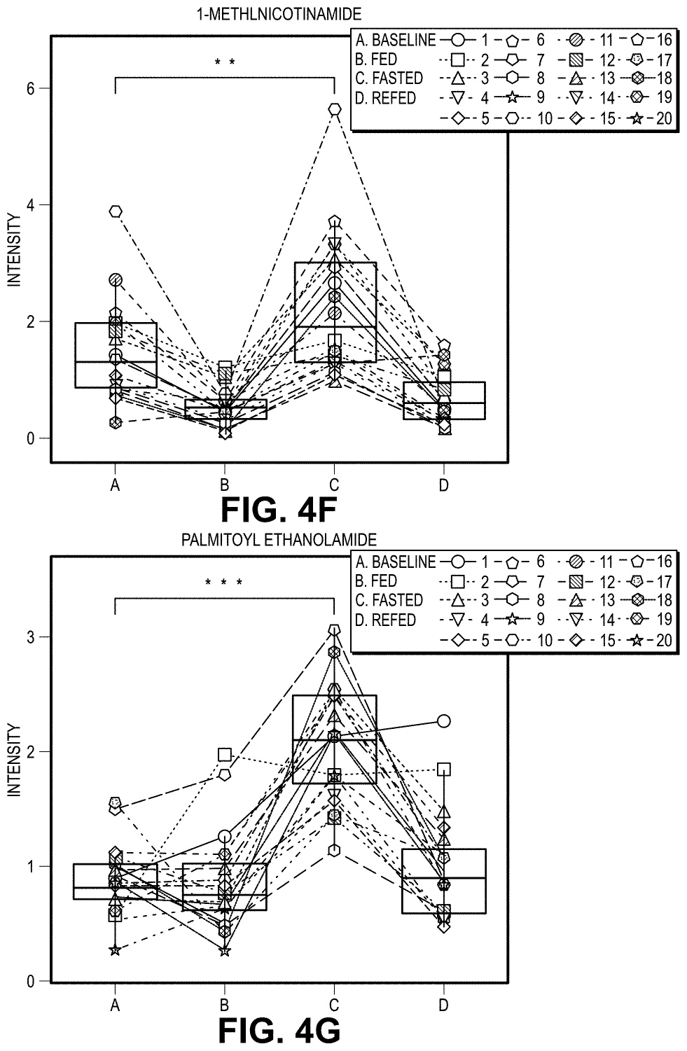

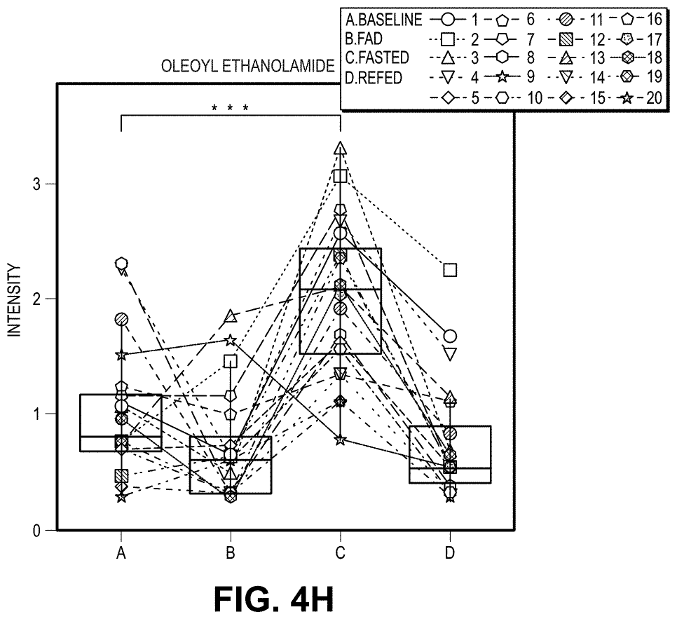

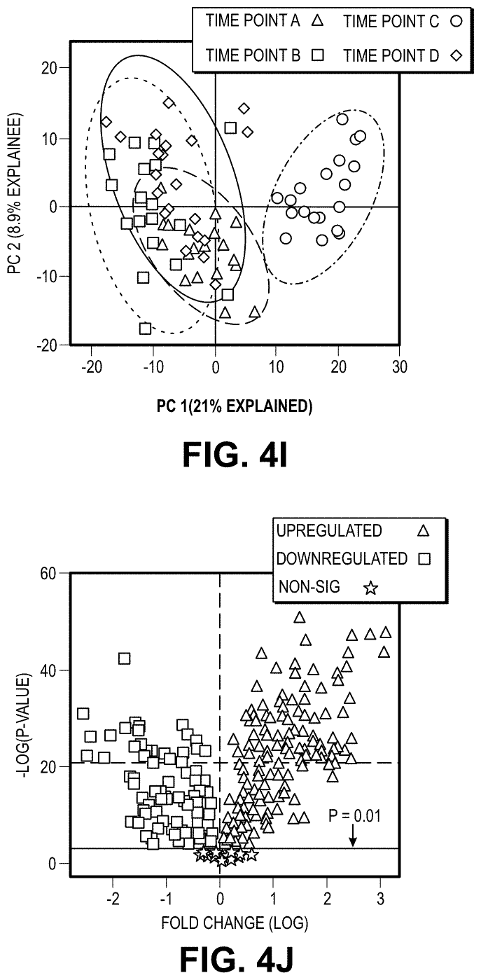

Comprehensive untargeted metabolomic analysis of participant plasma revealed remarkable alterations to the human metabolome in response to feeding, fasting, and refeeding (FIGS. 4A-4J). However, in order to delineate the distinct effects of PF rather than those achieved by the resolution of the postprandial response, analysis here was focused on differences observed between the Baseline and Fasted states. Even between the Baseline and Fasted states, there were more than 375 significantly differentially regulated metabolites even after adjustments for multiple comparisons (FIG. 4J) and PCA analysis revealed differences between the Fasted state and all other states (FIG. 41). Moreover, significant and consistent responses in multiple metabolites during PF were found as highlighted by the data of three of the most quantitatively altered metabolites 3-hydroxybutyrylcarnitine, acetoacetate, and the known anti-inflammatory ketone body beta-hydroxybutryate (BHB)³⁸, indicating multiple obligate responses to acute fasting in humans (FIGS. 4B and 4D). However, even within these obligate responses there was also considerable interindividual variability in the magnitude of response between participants by as much as 14× between the highest and lowest levels of an individual metabolite between participants in the Fasted state (FIG. 4C). This data underscores the importance of assessing and characterizing interindividual variability and how PF may contribute to differential functional responses between individuals and across states. Pathway analysis of the most differentially affected pathways between the Baseline and Fasted states revealed alterations to multiple fatty acid and amino acid biosynthesis and degradation pathways, ketone body metabolism, the tricarboxylic acid cycle, and nicotinamide metabolism (FIG. 4A). In agreement with a recent trial of long-term human alternate day fasting¹³, this study also found highly increased levels of immunomodulatory polyunsaturated fatty acids in the Fasted state indicating the potential involvement of altered fatty acid and eicosanoid signaling in the mechanisms underlying the effects of Fasted plasma on macrophage functionality (FIG. 4A). Ultimately, PF induced wide spread, highly significant, and, in some cases, universal alterations to the human metabolome beyond what is achieved during a typical overnight fast. Given these stark modulations, it was sought to assess how differences in plasma metabolites across states may play a key role in mediating the enhanced plasma functionalities and inducible immunomodulatory effects observed in the Fasted state.

### Example 8. Pf Upregulates Multiple Immunomodulatory Metabolites

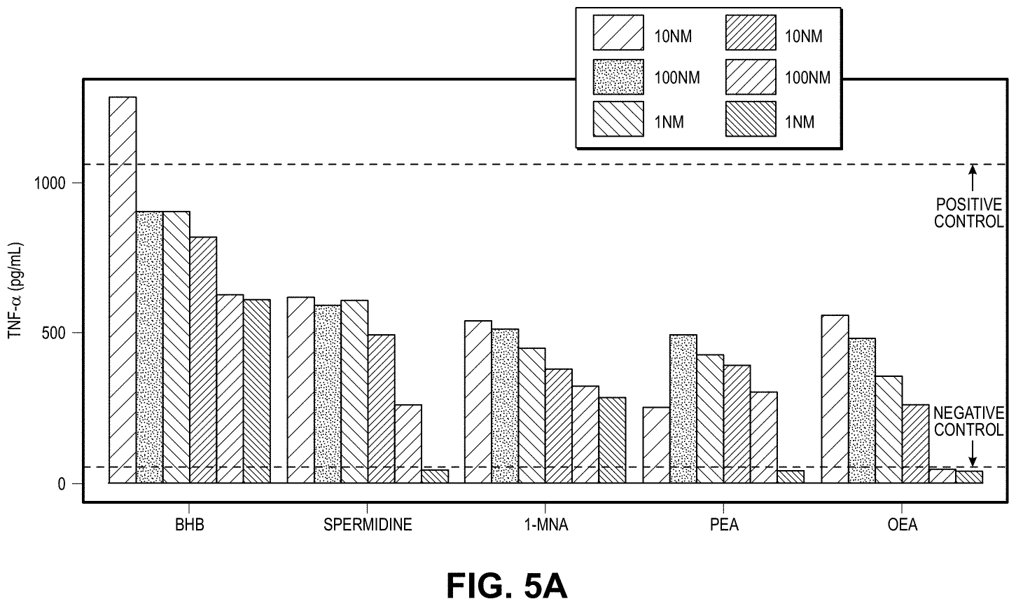

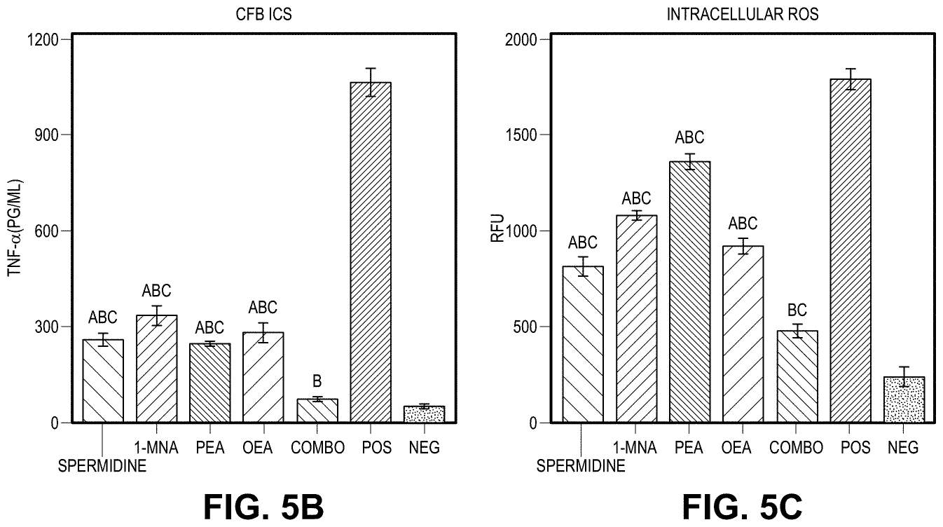

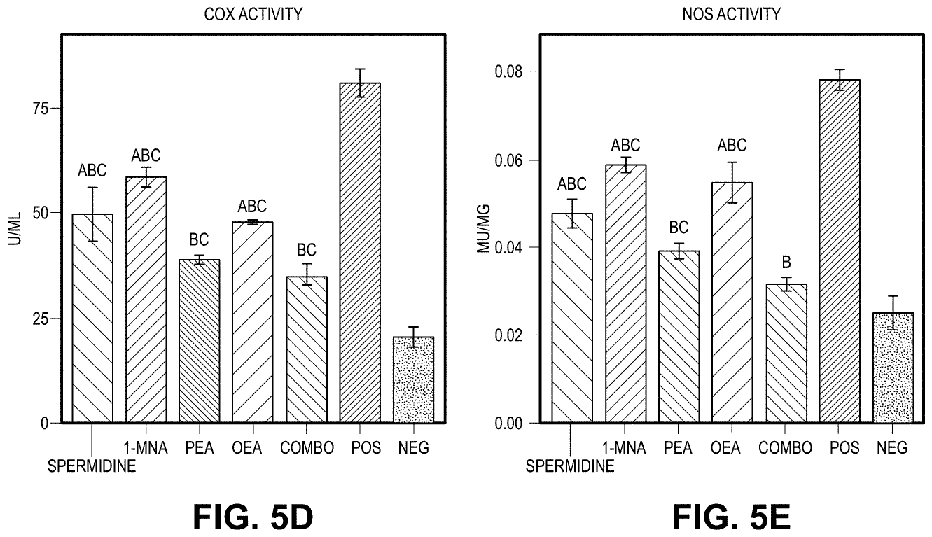

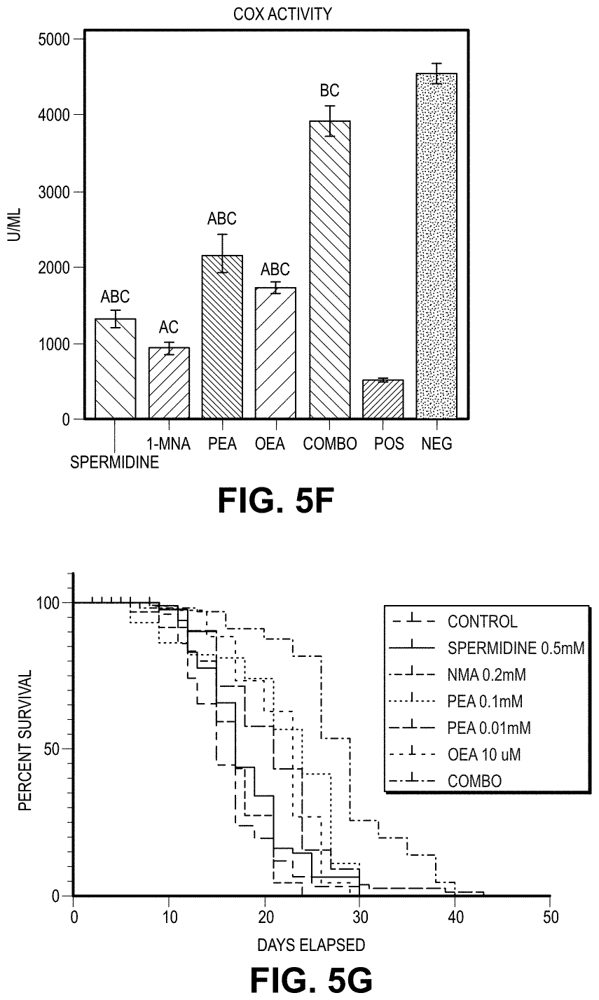

In order to determine the potential plasma borne mediators of PF's observed anti-inflammatory effects, the dataset of significantly upregulated metabolites between the Fasted and Baseline states was analyzed to find metabolites that had previously been shown to have effects on immune cell functionality. While BHB was highly upregulated during PF (FIG. 4B) and, due to its known anti-inflammatory effects, is often thought to be the major mediator of the immunomodulatory effects of PF^{38,39}, numerous other metabolites with known immunomodulatory effects that were upregulated in the Fasted state were also identified. Upon screening of these metabolites at various concentrations in an in vitro model of autoimmune disease as described above, it was found that 4 of the selected compounds: spermidine, 1-MNA, PEA, and OEA (FIGS. 4E-4H) were able to ameliorate TNF-α secretion from stimulated macrophage by at least 50% or more in comparison to treatment with 1 mM BHB (FIG. 5A).

Based on these results, these four metabolites were further investigated both alone and in combination (Combo) in the same in vitro functional analyses that was observed to be affected by treatment with Fasted plasma. For each metabolite, a single concentration was chosen to be tested based on the half maximal inhibitory concentration (IC50) of each metabolite during initial screening. Experimental results for each analysis are given for the following concentrations, Spermidine (100 μM), 1-MNA (100 μM), PEA (10 nM), OEA (10 μM), and Combo (100 μM spermidine, 100 μM 1-MNA, 10 nM PEA, 10 μM OEA).

### Example 9. Fasting Metabolites Replicate Anti-Inflammatory Effects of Fasted Plasma in Macrophage

As with Fasted plasma, treatment of primary human macrophage with individual compounds or their combination during stimulation with cFb ICs significantly reduced the secretion of TNF-α versus the vehicle treated positive control (FIG. 5B). Furthermore, the combination of metabolites together reduced TNF-α levels to a greater extent than any metabolite alone and almost to the level of the unstimulated negative control, indicating potent additive anti-inflammatory effects of the metabolites in combination. Similarly, all individual metabolites and their combination were capable of significantly reducing cellular ROS production from primary human macrophage and the level of ROS production in combination treated cells was significantly reduced beyond any of the metabolites alone (FIG. 5C). This result was also the case for total COX activity in LPS stimulated THP-1 macrophage where each individual compound and the combination treatment were able to significantly decrease the total COX activity versus the vehicle treated positive control (FIG. 5D). PEA was a potent inhibitor of COX activity and while combination treatment showed reduced levels of COX activity versus PEA, this was not found to be significantly lower than PEA alone (FIG. 5D).

This data underscores the potential of these metabolites to be responsible, at least in part, for the observed COX activity reduction in Fasted plasma. Finally, it was found that in vitro treatment of THP-1 macrophage with individual metabolites and their combination during induced M1 polarization showed a significant reduction in NOS activity with a corresponding increase in arginase activity versus the vehicle treated positive controls (FIGS. 5E and 5F). Decreases in NOS activity were observed to be significantly greater in Combo treated cells for all metabolites except PEA treated cells (FIG. 5E). Concomitant to blunted NOS activity, Combo treated cells also showed significantly higher arginase activity than all individual metabolites (FIG. 5F). Ultimately, these findings indicate that treatment of human macrophage with spermidine, 1-MNA, PEA, and OEA, all of which are upregulated in the Fasted state, are capable of replicating the immunomodulatory effects observed to be induced by Fasted plasma. Furthermore, treatment with a combination of these metabolites provided additional benefits to certain functional metrics, including significantly reduced TNF-α secretion from macrophage in an in vitro autoimmune disease model, significantly reduced cellular ROS production, and significantly increased arginase activity during induced M1 polarization. Therefore, spermidine, 1-MNA, PEA, and OEA may be important molecular mediators of at least a portion of the beneficial immunomodulatory effects of PF especially when used in combination.

### Example 10. Fasting Metabolites Extend Lifespan in C. Elegans

Beyond immunomodulation, PF has also been shown to significantly extend lifespan in model organisms. In order to assess the potential involvement of spermidine, 1-MNA, PEA, and OEA in mediating the lifespan extending effects of PF, lifespan analysis of C. elegans was performed through lifelong treatment with individual metabolites and their combination. Strikingly, it was found that treatment with spermidine, PEA, OEA and a combination of spermidine, 1-MNA, PEA, and OEA (Combo) all showed significantly increased lifespan extension versus untreated control worms (FIG. 5G). Of the individual metabolites, PEA and OEA treatments were found to have the greatest effect on lifespan extension and significantly increased lifespan versus spermidine treated worms (FIGS. 5A-5G). Importantly, a lower concentration of PEA was found to be the most effective at increasing lifespan indicating that PEA, while still beneficial at either concentration, has a point of diminishing returns that could be assessed when determining proper dosing for maximal lifespan extension in model organisms. This is the first trial to show that PEA and OEA are capable of extending lifespan in C. elegans. Similar to the results seen in vitro, the Combo treated worms showed the highest lifespan extension overall with lifespan being significantly (p<0.0001) increased from the Control group with a 100% increase to median lifespan and a 50% increase to maximal lifespan. Importantly, lifespan in Combo treated worms was also significantly (p<0.0001) increased in comparison to both PEA and OEA groups showing a powerful synergistic effect of the combination of these four metabolites to extend lifespan. These data are sufficient to indicate that spermidine, 1-MNA, PEA and OEA are all potential mediators of the lifespan extending effects of PF and furthermore that treatment with these fasting metabolites even under normal feeding conditions can induce fasting-like benefits to lifespan extension.

PF is capable of dramatically altering human plasma functionalities and the plasma metabolome, and treatment of non-fasted macrophage with Fasted plasma is capable of producing significant anti-inflammatory effects, and these effects are mediated, at least in part, by specific bioactive metabolites that are upregulated after 36 hours of fasting, and that these metabolites are capable of extending lifespan in C. elegans.

A key advantage of the current study was the rigorous and controlled design of the human trial. Unlike other studies of fasting, dietary intake within individuals was controlled using a controlled habitual diet, assessed compliance to fasting through personal glucose monitoring, and collected four distinct nutritional states from each individual across the study timecourse. These measures allowed the ability to sensitively assess differences in not only a 36 hr fasted state versus a postprandial state, as most other fasting trials have done, but also between a 36 hr fasted state and an overnight fasted state and postprandial states both before and after 36 hrs of fasting. This was critical in determining the distinct effects and mediators of PF beyond what is achieved during a typical overnight fast and the carry-over effects of PF into the Refed state without confounding dietary influences. Both of these outcomes are vitally important to the field of fasting research to help better understand how to make health and lifestyle recommendations on the duration and frequency of fasting required to achieve specific outcomes.

In this study, PF was capable of significantly increasing plasma antioxidant capacity as well as the cholesterol efflux ability of participant plasma over both the Baseline and Fed states and, furthermore, these increases carried over to the Refed state. Treatment of non-fasted macrophage with Fasted plasma also induced powerful anti-inflammatory effects significantly reducing TNF-α secretion, ROS production, M1 polarization responses, and total COX activity of stimulated human macrophage versus the Baseline and Fed states. These are the first studies to show that PF is capable of beneficially modifying human plasma functionalities and that treatment of human macrophage with Fasted plasma can induce significant anti-inflammatory effects beyond what is achieved by an overnight fast.

This is the first study to show that PF is also capable of ameliorating Fc-γ receptor induced macrophage activation and reducing total COX activity in macrophage, further elucidating the molecular pathways involved in PF's immunomodulatory effects. Additionally, PF is also capable of acutely decreasing M1 polarization responses in human macrophage. These analyses help to underscore that treatment of cells with participant plasma during in vitro analyses can induce cellular responses akin to what would be expected in vivo. Thus, such analyses can provide sensitive and real-time readouts of systemic functionality throughout the course of a clinical study. Taken together, these findings help to create a clearer picture of PF's systemic and cellular effects in humans and elucidate new molecular mechanisms underpinning these effects, specifically, the involvement of the Fc-γ and COX signaling pathways. Significantly, these results also indicate that not only does PF create anti-inflammatory effects in human macrophage, but that these effects are inducible in non-fasted cells via plasma-borne factors that may be responsible for mediating cellular responses to PF.

The importance of investigating these potential plasma-borne mediators cannot be understated as they represent a direct pathway by which the beneficial effects of PF may be replicated without the need to fast. The discovery of such mediators and their development into fasting mimetics represents enormous potential as therapeutic or preventative interventions for health and disease, especially in populations for whom fasting would be unsafe or undesirable. In the investigations of these potential mediators, it was showed that the human plasma metabolome in the Fasted state versus the Baseline state was remarkably altered with 389 metabolites being significantly differentially regulated between the two states, some with p values on the scale of 1×10⁻²⁰. Further analysis and screening of significantly upregulated metabolites in the Fasted state revealed spermidine, 1-MNA, PEA, and OEA to have significant anti-inflammatory effects on stimulated macrophage even beyond the effects of BHB at physiologically relevant doses reflective of those achievable with PF, which is often considered to be the major immunomodulatory metabolite during PF^{38,39}.

Assessment of these metabolites and their combination showed that all were able to replicate the anti-inflammatory effects observed in Fasting plasma including reductions in TNF-α secretion, ROS production, total COX activity, and M1 polarization responses in stimulated human macrophage. Furthermore, the combination of these metabolites induced the greatest reductions in all of these measures and was able to induce significantly lower TNF-α secretion and ROS production and significantly higher arginase activity, a marker of M2 polarization⁴⁶, than any of the individual metabolites alone. Thus, the upregulation of these metabolites appears to be responsible, at least in part, for the anti-inflammatory effects observed in Fasted plasma and, furthermore, a combination of these metabolites can be used to achieve similar functional effects as PF even in non-fasted cells.

This is the first trial to show that spermidine, 1-MNA, PEA, and OEA are distinctly upregulated in humans during PF. Moreover, this is the first trial to show their involvement in the immunomodulatory effects of PF on human innate immunity. Importantly, it was also showed that lifelong treatment of C. elegans with spermidine, PEA, OEA, and a combination of spermidine, 1-MNA, PEA, and OEA all significantly enhanced lifespan versus control worms even under normal feeding conditions. These results implicate the importance of these metabolites in mediating PF's well-known lifespan extending effects and further exemplify the potential for these compounds to be used as fasting mimetics or fasting enhancers in individuals for whom PF induces a lower magnitude of change in these metabolites. Significantly, this is also the first trial to show that PEA has lifespan extending effects and thus represents the discovery of a novel molecule for investigation in longevity research.

In conclusion, the results of this trial identified metabolites and mechanisms of action needed to develop future interventions designed to mimic the effects of PF. This study revealed new functionalities and pathways affected by PF in humans, identified four candidates for use as fasting mimetics, shown their effects to be most potent when used in combination, and discovered a previously unknown role of PEA in lifespan extension. Human supplementation with spermidine, 1-MNA, PEA, and OEA both alone and in combination may be able to enhance the effects of fasting, reducing the duration or frequency of fasting required to achieve effects, mimicking the effects of fasting in populations who are unable to fast, and reducing pro-inflammatory activation in the postprandial state. Additionally, the results also underscore the effectiveness of this controlled study design as a platform for characterizing individual differences in response to different fasting regimens as well as the discovery of novel mediators of PF-induced effects on cellular functionality.

### Example 11. Clinical Fasting Method for the Study of Native Microbiome Dynamics

The human microbiome is a vast and highly complex ecosystem with far reaching health implications from metabolism to disease progression to cognitive functionality. While the intestinal microbiome can be heavily influenced by dietary intake and food choices, the effects of the absence of nutritional intake (as experienced during prolonged fasting) on the microbiome have yet to be determined. This is especially of interest as the study of the microbiome without the influence of exogenous dietary influences may reveal the so called “native” microbiome consisting of populations of micro-organisms selected and sustained by the influences of the human body alone (i.e., immune system responses, mucin secretion, etc.) rather than by the influence of food choices. Furthermore, the removal of dietary influences on the microbiome allows for the creation of a baseline framework of native microbial function against which targeted interventions (i.e., probiotics, supplements, food products, etc.) can be studied in isolation for their specific effects on microbiome dynamics without the confounding influence of other exogenous dietary factors. Such experiments would pave the way to a precise understanding of how individual foods, food components, and/or supplements influence the microbiome. The present study established a clinical study design including a period of 36 hours of zero-calorie fasting for the creation of a native baseline microbiome state free from exogenous dietary influences. It was shown that 36 hours of fasting induces massive changes to circulating intestinally derived microbial metabolites and establish a framework of metabolites that can be used to assess native vs exogenous microbial activities.

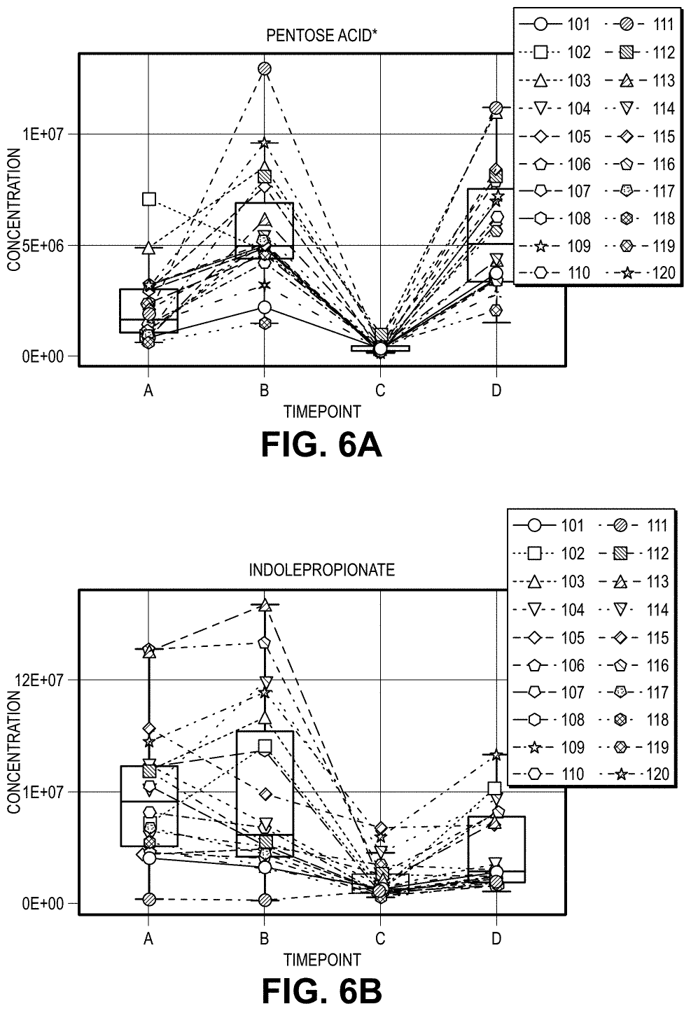

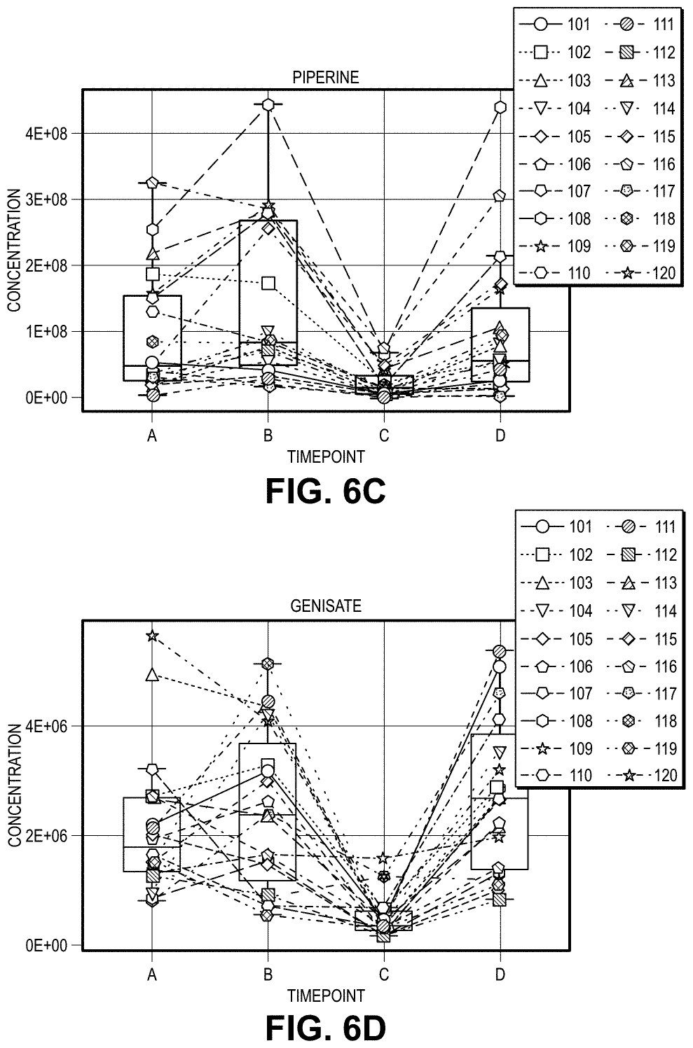

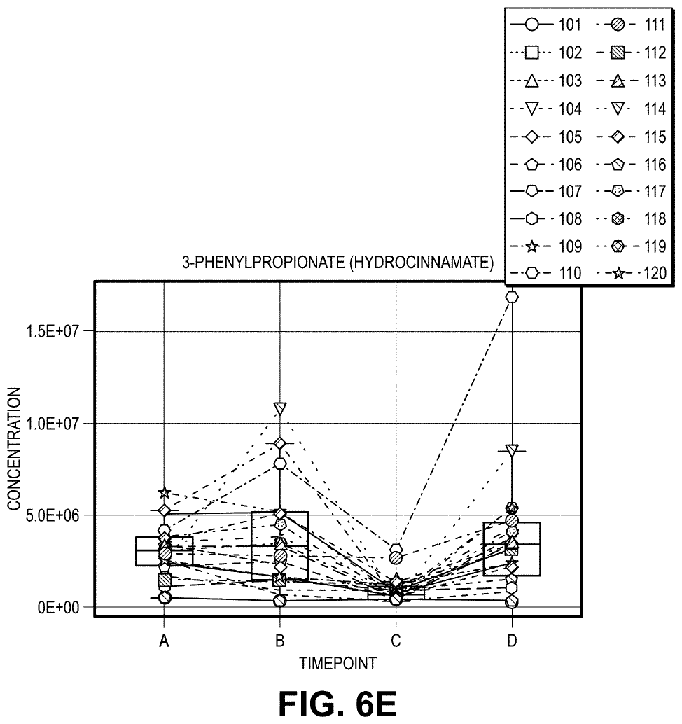

To highlight the impact of complete dietary restriction on the metabolism and functionality of the intestinal microbiome, a human clinical trial of 36 hours of zero-calorie fasting in 20 young healthy individuals including a Baseline overnight fasted state, a 36 hr Fasted state, a postprandial Fed state, and a second postprandial Refed state after 36 hr of fasting was conducted. Massive changes to circulating levels of microbial metabolites in human plasma between not only the Fed and Fasted states but also the Baseline and Fasted states were observed, which underscores the dynamic and time-sensitive process of microbial metabolism. The major effect of fasting on the microbiome seems to be the suppression of microbial metabolite synthesis and circulation. This is in line with the lack of dietary intake as many microbial metabolites require exogenous precursors that are either absent or highly depleted during fasting. Of the numerous metabolites found to be downregulated during fasting, several were shown to be universally depleted among all participants almost to the point of non-detection including pentose acid, indolepropionate, piperine, gentisate, and hydrocinnamate (FIGS. 6A-6E). The depletion of these metabolites is only achieved in the Fasted state and is not observed in the Baseline state indicating that a simple overnight fast is not sufficient to completely remove the influence of dietary intake on microbial metabolism. Furthermore, the levels of these metabolites, while depleted during fasting, are rapidly regenerated after dietary intake is resumed. The levels of pentose acid, gentisate, piperine, and hydrocinnamate in the Refed state are all non-significantly different than the initial Fed state underscoring the sensitivity and responsiveness of these metabolites to dietary intake.

Based on this, the depletion of these metabolites can be used as sensitive markers of prolonged fasting and as real-time plasma-borne indicators of the shift in microbial metabolism from exogenous dietary sources from food intake to endogenous energy sources from mucins and microbial breakdown products.

The depletion of microbial metabolites during prolonged fasting, as observed with pentose acid, indolepropionate, gentisate, piperine, and hydrocinnamate, offers an exciting framework for the study of the impact of individual targeted interventions on microbial metabolism and functionality. The results demonstrate that the clinical study design using a controlled habitual diet and 36 hrs of zero calorie fasting is capable of creating such a framework via the removal of dietary influences on the intestinal microbiome and microbial metabolism that cannot be achieved through a simple overnight fast. Furthermore, the depletion of the metabolites pentose acid, indolepropionate, gentisate, piperine, and hydrocinnamate can serve as measures of compliance for fasting as well as easily measurable plasma indicators of shifts in microbial metabolism from exogenous sources to endogenous sources. The removal of dietary influence on the microbiome is of interest to the microbiome research field as it allows investigators the ability to study the effects of individual interventions (i.e., foods, food components, supplements, pro-/prebiotics, drugs, etc.) on the microbiome in isolation of confounding nutritional factors.

### Example 12

In order to assess the bioavailability of spermidine, 1-MNA, palmitoylethanolamide (PEA) and oleoylethanolamide (OEA) and their combined ability to induce similar beneficial effects as fasting and as observed in vitro and in model organisms, a small (n=5) clinical pilot study was performed of oral supplementation with a combination of these metabolites in young healthy people. Briefly, five healthy young men (Age: 25±2.7, BMI:21.4±3.1) underwent four arms of a repeated measures clinical study design consisting of four single study day protocols assessing the effects of supplementation with an escalating dose of a combination spermidine, nicotinamide, PEA, and OEA along with consumption of a standardized breakfast. This supplement combination will be referred to as FM-01. At the start of each study day, participants ate a standardized breakfast consisting of 2 Larabars (440 Calories; Ingredients: Dates and Cashews) at approximately 6 AM. 2 hours after consuming the standardized breakfast participants provided a postprandial blood sample (TO) and then were orally supplemented with FM-01 during a four different dosing arms Low, Medium, or High dose (Low: 5 g of wheat germ extract standardized to an equivalent of 5 mg of spermidine, 250 mg of nicotinamide, 400 mg of PEA, 200 mg of OEA; Medium: 10 g of wheat germ extract standardized to an equivalent of 10 mg of spermidine, 500 mg of nicotinamide, 800 mg of PEA, 400 mg of OEA High: 15 g of wheat germ extract standardized to an equivalent of 15 mg of spermidine, 750 mg of nicotinamide, 1200 mg of PEA, 600 mg of OEA) or given no supplementation with FM-01 (Control) with a one week washout period between each dosing arm. Additional blood samples were then provided at 1 hr (T1), 2 hr (T2) and 4 hr (T4) after supplementation with FM-01 (Low, Medium, High) or after the baseline TO time point (Control). Blood samples were immediately processed to yield participant plasma and stored at −80 C. Plasma samples from each timepoint and each participant were experimentally assessed for their anti-inflammatory ability, antioxidant ability, and cholesterol efflux ability using in vitro cellular assay with primary human macrophage as described above.

Statistics: Statistical analysis for each in vitro functional assessment was performed in Prism 7 and significance (p<0.05) vs baseline TO timepoints for each arm were determined using repeated measures ANOVAs with corrections for multiple comparison.

### Results

### Plasma Anti-Inflammatory Ability

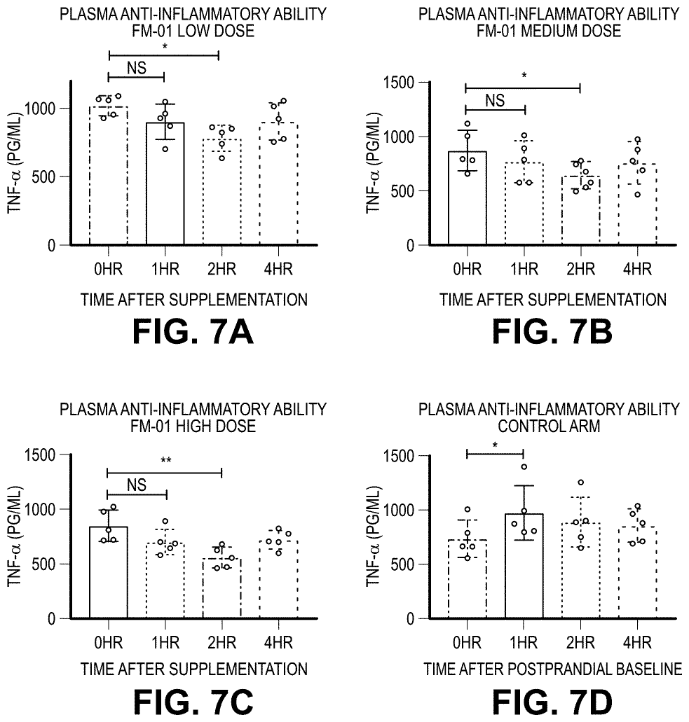

Consumption of the standardized breakfast alone (Control Arm) caused a significant decrease in the ability of subject plasma to reduce TNF-α secretion from cFb IC stimulated primary macrophage versus the T0 baseline timepoint at the T1 timepoint. See, FIG. 7A-D. This is typical and indicative of the well-known inflammatory postprandial response that accompanies food consumption. Supplementation with FM-01 at all dosage levels was able to significantly increase the ability of subject plasma to reduce TNF-α secretion from stimulated primary human macrophage versus the T0 baseline timepoint at the T2 timepoint. This effect was greatest in the High Dose arm. Importantly, supplementation with FM-01 at all dosages was also able to prevent the significant loss of plasma anti-inflammatory ability at the T1 timepoint observed in the Control Arm indicating that supplementation with FM-01 creates anti-inflammatory plasma effects within 1 hour of supplementation which continues into the T2 timepoint. These results indicate that supplementation with FM-01, even at the Low Dose, can help to prevent postprandial dietary inflammation and create significant anti-inflammatory effects just hours after supplementation even when taken with food.

### Plasma Antioxidant Ability

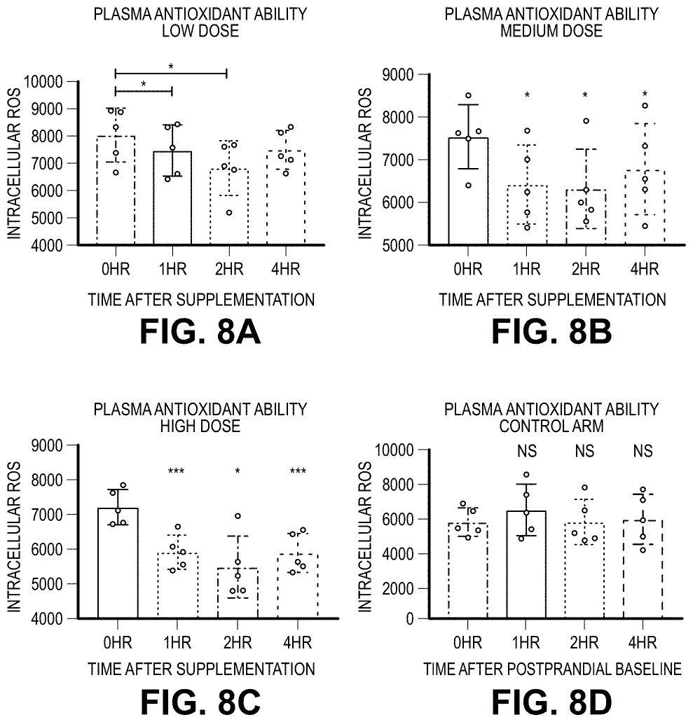

Consumption of the standardized breakfast alone (Control Arm) did not cause any significant changes in the ability of subject plasma to modify the abundance of intracellular ROS in primary macrophage exposed to 250 uM TBHP. See, FIG. 8A-D. Conversely, supplementation with FM-01 at all dosage levels significantly increased subject plasma antioxidant ability as seen by lower accumulation of intracellular ROS at the T1 and T2 timepoint versus the TO baseline state. This significant increase in plasma antioxidant ability was abrogated at the T4 timepoint in the Low Dose arm, but was sustained through the T4 timepoint for both the Medium and High dose arms. These results indicate that FM-01 is able to induce significant improvements to the cytoprotective antioxidant ability of human plasma and, at higher doses, can create sustained benefits until at least 4 hours after supplementation even when taken with food.

### Plasma Cholesterol Efflux Ability

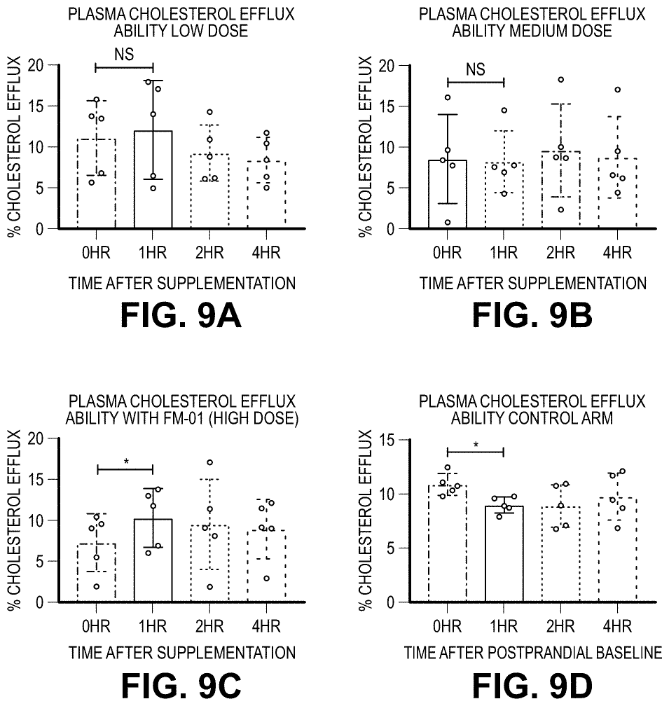

Consumption of the standardized breakfast alone (Control Arm) caused a significant decrease in subject plasma cholesterol efflux ability from lipid-load primary macrophage in the T1 timepoint vs the T0 baseline timepoint, but not in the T2 or T4 timepoints. See, FIG. 9A-D. Conversely, supplementation with FM-01 at all doses was able to prevent this significant loss in plasma cholesterol efflux ability in the T1 timepoint and, strikingly, was able to significantly improve plasma cholesterol efflux ability in the High Dose arm. The T2 and T4 timepoints for all dosage levels was not significantly different than the TO baseline timepoint. These results indicate that supplementation with FM-01 is able to prevent a postprandial loss of plasma cholesterol efflux ability and, impressively, at high doses significantly improve plasma cholesterol efflux ability even in the postprandial state. As plasma cholesterol efflux ability is the most predictive clinical measure for cardiovascular disease risk, supplementation with FM-01 may be able to have a significant impact on CVD risk and improve cardiovascular health by helping to ameliorate the negative effects of improper nutrition.

### REFERENCES

1. Longo, V. D. & Mattson, M. P. Fasting: Molecular mechanisms and clinical applications. Cell Metabolism (2014) doi:10.1016/j.cmet.2013.12.008.
2. Mattson, M. P., Longo, V. D. & Harvie, M. Impact of intermittent fasting on health and disease processes. Ageing Research Reviews (2017) doi: 10.1016/j.arr.2016.10.005.
3. Longo, V. D. & Panda, S. Fasting, Circadian Rhythms, and Time-Restricted Feeding in Healthy Lifespan. Cell Metabolism (2016) doi:10.1016/j.cmet.2016.06.001.
4. Lee, C. & Longo, V. Dietary restriction with and without caloric restriction for healthy aging. F1000Research (2016) doi:10.12688/f1000research.7136.1.
5. Longo, V. D. & Fontana, L. Calorie restriction and cancer prevention: metabolic and molecular mechanisms. Trends in Pharmacological Sciences (2010) doi:10.1016/j.tips.2009.11.004.
6. Ahmed, A. et al. Impact of intermittent fasting on human health: An extended review of metabolic cascades. Int. J. Food Prop. (2018) doi:10.1080/10942912.2018.1560312.
7. Malinowski, B. et al. Intermittent fasting in cardiovascular disorders—an overview. Nutrients (2019) doi:10.3390/nu11030673.
8. Tinsley, G. M. & La Bounty, P. M. Effects of intermittent fasting on body composition and clinical health markers in humans. Nutr. Rev. (2015) doi:10.1093/nutrit/nuv041.
9. de Azevedo, F. R., Ikeoka, D. & Caramelli, B. Effects of intermittent fasting on metabolism in men. Revista da Associacao Medica Brasileira (2013) doi:10.1016/j.ramb.2012.09.003.
10. Trepanowski, J. F. et al. Effect of alternate-day fasting on weight loss, weight maintenance, and cardioprotection among metabolically healthy obese adults: A randomized clinical trial. JAMA Intern. Med. (2017) doi:10.1001/jamainternmed.2017.0936.
11. Choi, I. Y., Lee, C. & Longo, V. D. Nutrition and fasting mimicking diets in the prevention and treatment of autoimmune diseases and immunosenescence. Mol. Cell. Endocrinol. (2017) doi:10.1016/j.mce.2017.01.042.
12. Adawi, M. et al. Ramadan fasting exerts immunomodulatory effects: Insights from a systematic review. Frontiers in Immunology (2017) doi:10.3389/fimmu.2017.01144.
13. Stekovic, S. et al. Alternate Day Fasting Improves Physiological and Molecular Markers of Aging in Healthy, Non-obese Humans. Cell Metab. (2019) doi:10.1016/j.cmet.2019.07.016.
14. Traba, J. et al. Fasting and refeeding differentially regulate NLRP3 inflammasome activation in human subjects. J. Clin. Invest. (2015) doi:10.1172/JCI83260.
15. Nagai, M. et al. Fasting-Refeeding Impacts Immune Cell Dynamics and Mucosal Immune Responses. Cell (2019) doi:10.1016/j.cell.2019.07.050.
16. Jordan, S. et al. Dietary Intake Regulates the Circulating Inflammatory Monocyte Pool. Cell (2019) doi:10.1016/j.cell.2019.07.050.
17. Lavin, D. N. et al. Fasting induces an anti-inflammatory effect on the neuroimmune system which a high-fat diet prevents. Obesity (2011) doi:10.1038/oby.2011.73.
18. Sun, P. et al. Fasting inhibits colorectal cancer growth by reducing M2 polarization of tumor-associated macrophages. Oncotarget (2017) doi:10.18632/oncotarget.20301.
19. Traba, J. et al. Prolonged fasting suppresses mitochondrial NLRP3 inflammasome assembly and activation via SIRT3-mediated activation of superoxide dismutase 2. J. Biol. Chem. (2017) doi:10.1074/jbc.M117.791715.
20. Jung, J., Zeng, H. & Horng, T. Metabolism as a guiding force for immunity. Nature Cell Biology (2019) doi:10.1038/s41556-018-0217-x.
21. O'Neill, L. A. J., Kishton, R. J. & Rathmell, J. A guide to immunometabolism for immunologists. Nature Reviews Immunology (2016) doi:10.1038/nri.2016.70.
22. Meessen, E. C. E., Warmbrunn, M. V., Nieuwdorp, M. & Soeters, M. R. Human postprandial nutrient metabolism and low-grade inflammation: A narrative review. Nutrients (2019) doi:10.3390/nu11123000.
23. Burdge, G. C. & Calder, P. C. Plasma cytokine response during the postprandial period: a potential causal process in vascular disease? Br. J. Nutr. (2005) doi:10.1079/bjn20041282.
24. Jeacocke, N. A. & Burke, L. M. Methods to standardize dietary intake before performance testing. International Journal of Sport Nutrition and Exercise Metabolism (2010) doi:10.1123/ijsnem.20.2.87.
25. Shalaurova, I., Connelly, M. A., Garvey, W. T. & Otvos, J. D. Lipoprotein insulin resistance index: A lipoprotein particle-derived measure of insulin resistance. Metab. Syndr. Relat. Disord. (2014) doi:10.1089/met.2014.0050.
26. MacKey, R. H. et al. Lipoprotein particles and incident type 2 diabetes in the multi-ethnic study of atherosclerosis. Diabetes Care (2015) doi:10.2337/dc14-0645.
27. Harada, P. H. N. et al. Lipoprotein insulin resistance score and risk of incident diabetes during extended follow-up of 20 years: The Women's Health Study. J. Clin. Lipidol. (2017) doi:10.1016/j.jac1.2017.06.008.
28. Connelly, M. A. et al. GlycA, a marker of acute phase glycoproteins, and the risk of incident type 2 diabetes mellitus: PREVEND study. Clin. Chim. Acta (2016) doi:10.1016/j.cca.2015.11.001.
29. Ormseth, M. J. et al. Utility of a novel inflammatory marker, GlycA, for assessment of rheumatoid arthritis disease activity and coronary atherosclerosis. Arthritis Res. Ther. (2015) doi:10.1186/s13075-015-0646-x.
30. Ford, L. et al. Precision of a Clinical Metabolomics Profiling Platform for Use in the Identification of Inborn Errors of Metabolism. J. Appl. Lab. Med. (2020) doi:10.1093/jalm/jfz026.
31. Dehaven, C. D., Evans, A. M., Dai, H. & Lawton, K. A. Organization of GC/MS and LC/MS metabolomics data into chemical libraries. J Cheminform. (2010) doi:10.1186/1758-2946-2-9.
32. Sokolove, J., Zhao, X., Chandra, P. E. & Robinson, W. H. Immune complexes containing citrullinated fibrinogen costimulate macrophages via toll-like receptor 4 and Fcγ receptor. Arthritis Rheum. (2011) doi:10.1002/art.30081.
33. Zajac, E. et al. Angiogenic capacity of M1- and M2-polarized macrophages is determined by the levels of TIMP-1 complexed with their secreted proMMP-9. Blood (2013) doi:10.1182/blood-2013-05-501494.
34. Ritchie, M. E. et al. Limma powers differential expression analyses for RNA-sequencing and microarray studies. Nucleic Acids Res. (2015) doi:10.1093/nar/gkv007.
35. Bates, D., Machler, M., Bolker, B. M. & Walker, S. C. Fitting linear mixed-effects models using lme4. J. Stat. Softw. (2015) doi:10.18637/jss.v067.i01.
36. Hothorn, T., Bretz, F. & Westfall, P. Simultaneous inference in general parametric models. Biometrical Journal (2008) doi:10.1002/bimj.200810425.
37. Kim, K. H. et al. Intermittent fasting promotes adipose thermogenesis and metabolic homeostasis via VEGF-mediated alternative activation of macrophage. Cell Res. (2017) doi:10.1038/cr.2017.126.
38. Youm, Y. H. et al. The ketone metabolite β-hydroxybutyrate blocks NLRP3 inflammasome-mediated inflammatory disease. Nat. Med. (2015) doi:10.1038/nm.3804.
39. Rojas-Morales, P., Tapia, E. & Pedraza-Chaverri, J. I3-Hydroxybutyrate: A signaling metabolite in starvation response? Cellular Signalling (2016) doi:10.1016/j.cellsig.2016.04.005.
40. Chlopicki, S. et al. 1-Methylnicotinamide (MNA), a primary metabolite of nicotinamide, exerts anti-thrombotic activity mediated by a cyclooxygenase-2/prostacyclin pathway. Br. J. Pharmacol. (2007) doi:10.1038/sj.bjp.0707383.
41. Choi, Y. H. & Park, H. Y. Anti-inflammatory effects of spermidine in lipopolysaccharide-stimulated BV2 microglial cells. J. Biomed. Sci. (2012) doi:10.1186/1423-0127-19-31.
42. Gabrielsson, L., Gouveia-Figueira, S., Haggstrom, J., Alhouayek, M. & Fowler, C. J. The anti-inflammatory compound palmitoylethanolamide inhibits prostaglandin and hydroxyeicosatetraenoic acid production by a macrophage cell line. Pharmacol. Res. Perspect. (2017) doi:10.1002/prp2.300.
43. Payahoo, L., Khajebishak, Y., Jafarabadi, M. A. & Ostadrahimi, A. Oleoylethanolamide supplementation reduces inflammation and oxidative stress in obese people: A clinical trial. Adv. Pharm. Bull. (2018) doi:10.15171/apb.2018.056.
44. Schmeisser, K. et al. Role of sirtuins in lifespan regulation is linked to methylation of nicotinamide. Nat. Chem. Biol. (2013) doi:10.1038/nchembio.1352.
45. Eisenberg, T. et al. Induction of autophagy by spermidine promotes longevity. Nat. Cell Biol. (2009) doi:10.1038/ncb1975.
46. Jha, A. K. et al. Network integration of parallel metabolic and transcriptional data reveals metabolic modules that regulate macrophage polarization. Immunity (2015) doi:10.1016/j.immuni.2015.02.005.
47. Madeo, F., Eisenberg, T., Pietrocola, F. & Kroemer, G. Spermidine in health and disease. Science (2018) doi:10.1126/science.aan2788.
48. Pietrocola, F. et al. Caloric Restriction Mimetics Enhance Anticancer Immunosurveillance. Cancer Cell (2016) doi:10.1016/j.ccell.2016.05.016.
49. Gewandter, J. S. et al. Reporting of cross-over clinical trials of analgesic treatments for chronic pain: Analgesic, Anesthetic, and Addiction Clinical Trial Translations, Innovations, Opportunities, and Networks systematic review and recommendations. Pain (2016) doi:10.1097/j.pain.0000000000000673.
50. Sabin, C. A. Clinical trials: a practical guide to design, analysis and reporting. Clin. Med. (Northfield. Il). (2006) doi:10.7861/clinmedicine.6-6-619a.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A composition comprising one or more of metabolites selected from the group consisting of spermidine, 1-methylnicotinamide (1-MNA), palmitoylethanolamide (PEA), and oleoylethanolamide (OEA) in an amount sufficient to elevate the circulating levels of the metabolites to a same level or higher than the circulating levels of the metabolites that are achieved through prolonged (e.g., at least 20 hours, e.g., 20-72 hours) fasting in a subject.

2. A composition comprising one or more of metabolites selected from the group consisting of spermidine, 1-methylnicotinamide (1-MNA), palmitoylethanolamide (PEA), and oleoylethanolamide (OEA) in an amount sufficient to induce an anti-inflammatory, anti-oxidant, and/or anti-apoptotic effect in a subject.

3. The composition of claim 1 or 2, comprising two of the metabolites.

4. The composition of claim 3, wherein the two metabolites are spermidine and 1-MNA, spermidine and PEA, spermidine and OEA, 1-MNA and PEA, 1-MNA and OEA, or PEA and OEA.

5. The composition of claim 1 or 2, wherein the composition comprises three of the metabolites.

6. The composition of claim 5, wherein the three metabolites are 1) sperimidine, 1-MNA, and PEA, 2) spermidine, 1-MNA, and OEA, 3) spermidine, PEA, and OEA, or 4) 1-MNA, PEA, and OEA.

7. The composition of claim 1 or 2, wherein the composition comprises all four of the metabolites.

8. A composition comprising one or more of metabolites selected from the group consisting of spermidine or a precursor thereof, 1-methylnicotinamide (1-MNA) or a precursor thereof, palmitoylethanolamide (PEA) or a precursor thereof, and oleoylethanolamide (OEA) or a precursor thereof in an amount sufficient to induce an anti-inflammatory, anti-oxidant, and/or anti-apoptotic effect in a subject.

9. A composition comprising one or more of metabolites selected from the group consisting of spermidine or a precursor thereof, 1-methylnicotinamide (1-MNA) or a precursor thereof, palmitoylethanolamide (PEA) or a precursor thereof, and oleoylethanolamide (OEA) or a precursor thereof in an amount sufficient to elevate the circulating levels of the metabolites to a same level or higher than the circulating levels of the metabolites that are achieved through prolonged (e.g., at least 20 hours, e.g., 20-72 hours) fasting in a subject.

10. The composition of claim 8 or 9, comprising two of the metabolites or a precursor thereof.

11. The composition of claim 10, wherein the two metabolites are spermidine or a precursor thereof and 1-MNA or a precursor thereof, spermidine or a precursor thereof and PEA or a precursor thereof, spermidine or a precursor thereof and OEA or a precursor thereof, 1-MNA or a precursor thereof and PEA or a precursor thereof 1-MNA or a precursor thereof and OEA or a precursor thereof, or PEA or a precursor thereof and OEA or a precursor thereof.

12. The composition of claim 8 or 9, wherein the composition comprises three of the metabolites or a precursor thereof.

13. The composition of claim 12, wherein the three metabolites are 1) sperimidine or a precursor thereof, 1-MNA or a precursor thereof, and PEA or a precursor thereof, 2) spermidine or a precursor thereof or a precursor thereof, 1-MNA or a precursor thereof, and OEA or a precursor thereof, 3) spermidine or a precursor thereof, PEA or a precursor thereof, and OEA or a precursor thereof, or 4) 1-MNA or a precursor thereof, PEA or a precursor thereof, and OEA or a precursor thereof.

14. The composition of claim 8 or 9, wherein the composition comprises all four of the metabolites or a precursor thereof.

15. The composition of any one of claims 1-14, wherein the composition comprises a precurser of 1-MNA selected from the group consisting of nictotinamide, niacinamide, and nicotinamide riboside.

16. The composition of any one of claims 1-14, wherein the precurser of PEA is palmitic acid.

17. The composition of any one of claims 1-14, wherein the precurser of OEA is oleic acid.

18. The composition of any one of claims 1 to 14, wherein the ratio of two, three, or four of the metabolites is about 10000:1000:1:1000 of spermidine:1-MNA:PEA:OEA (w:w:w:w).

19. The composition of any one of claims 1 to 14, wherein the composition comprises 5-15 mg spermidine, 400-1200 mg PEA, 300-600 mg OEA, and 500-1000 mg nicotinamide.

20. The composition of any one of claims 1 to 18, wherein the composition is formulated as a dietary supplement.

21. The composition of any one of claims 1 to 20, wherein the composition is formulated for oral administration.

22. The composition of claim 21, wherein the composition is formulated as a pill, a tablet, gummy, sublingual, spray, candy, nutrition bar, energy shot, beverage, or a syrup.

23. The composition of any one of claims 1 to 20, wherein the composition is formulated for intravenous administration, transdermal administration or topical administration.

24. A method for inducing an anti-inflammatory, anti-oxidant, and/or anti-apoptotic effect in a subject, comprising administering to the subject one or more of metabolites selected from the group consisting of spermidine or a precursor thereof, 1-methylnicotinamide (1-MNA) or a precursor thereof, palmitoylethanolamide (PEA) or a precursor thereof, and oleoylethanolamide (OEA) or a precursor thereof in an amount sufficient to induce the anti-inflammatory, anti-oxidant, and/or anti-apoptotic effect in the subject.

25. The method of claim 24, wherein the method decreases the amount of tumor necrosis factor alpha (TNF-α) secreted by macrophages in the subject relative to the amount of TNF-α secreted by macrophages in the subject prior to the subject receiving the metabolite.

26. The method of claim 25, wherein the amount of TNF-α secreted by macrophages after the subject receiving the metabolite is less than 90% of the amount of TNF-α secreted by macrophages prior to the subject receiving the metabolite.

27. The method of any one of claims 24 to 26, wherein the method increases total antioxidant capacity of the subject's plasma relative to the total antioxidant capacity of the subject's plasma prior to the subject receiving the metabolite.

28. The method of any one of claims 24 to 27, wherein the method increases cholesterol efflux of the subject relative to the cholesterol efflux of the subject prior to the subject receiving the metabolite.

29. The method of any one of claims 24 to 28, wherein the method decreases the amount of reactive oxygen species (ROS) produced by macrophages in the subject relative to the amount of ROS produced by macrophages in the subject prior to the subject receiving the metabolite.

30. The method of any one of claims 24 to 29, wherein the method decreases cyclooxygenase (COX) activity in the subject relative to the COX activity in the subject prior to the subject receiving the metabolite.

31. The method of any one of claims 24 to 30, wherein the method decreases nitric oxide synthases (NOS) activity in the subject relative to the NOS activity in the subject prior to the subject receiving the metabolite.

32. The method of any one of claims 24 to 31, wherein the method decreases macrophage M1 polarization in the subject relative to the macrophage M1 polarization in the subject prior to the subject receiving the metabolite.

33. The method of any one of claims 24 to 32, wherein the method increases arginase activity in the subject relative to the arginase activity in the subject prior to the subject receiving the metabolite.

34. The method of any one of claims 24 to 33, wherein the method increases macrophage M2 polarization in the subject relative to the macrophage M2 polarization in the subject prior to the subject receiving the metabolite.

35. The method of any one of claims 24 to 34, wherein the method extends longevity and/or improves cognitive and/or physical performance of the subject.

36. The method of any one of claims 24 to 35, wherein the subject is on a fasting diet.

37. The method of claim 36, wherein one or more of metabolies selected from the group consisting of pentose acid, indolepropionate, gentisate, piperine, and hydrocinnamate are substantially depleted in the subject.

38. The method of any one of claims 24 to 37, wherein the subject has an inflammatory disorder.

39. The method of claim 38, wherein the inflammatory disorder is selected from the group consisting of rheumatoid arthritis (RA), systemic lupus erythematosus (SLE), ANCA-associated vasculitis, antiphospholipid antibody syndrome, autoimmune hemolytic anemia, chronic inflammatory demyelinating neuropathy, graft-vs-host disease (GVHD), dermatomyositis, Goodpasture's Syndrome, organ system-targeted type II hypersensitivity syndromes, Guillain Barre syndrome, chronic inflammatory demyelinating polyneuropathy (CIDP), dermatomyositis, Felty's syndrome, autoimmune thyroid disease, ulcerative colitis, autoimmune liver disease, idiopathic thrombocytopenia purpura, Myasthenia Gravis, neuromyelitis optica, pemphigus, Sjogren's Syndrome, autoimmune cytopenias, synovitis, dermatomyositis, systemic vasculitis, glomerulitis, and vasculitis.

40. The method of any one of claims 24 to 38, wherein the subject has a metabolic disorder.

41. The method of claim 40, wherein the metabolic disorder is selected from the group consisting of non-alcoholic fatty liver disease (NAFLD), non-alcoholic steatohepatitis (NASH), diabetes, and metabolic syndrome.

42. The method of any one of claims 24 to 40, wherein the subject is overweight.

43. The method of any one of claims 24 to 42, wherein the metabolite is administered to the subject one or more times daily.

44. The method of any one of claims 24 to 43, wherein the metabolite is administered to the subject during food intake.

45. The method of any one of claims 24 to 44, wherein the metabolite is administered to the subject after at least 5 hours of fasting.

46. The method of any one of claims 24 to 45, wherein the method comprises administering two of the metabolites.

47. The method of claim 46, wherein the two metabolites are spermidine or a precursor thereof and 1-MNA or a precursor thereof, spermidine or a precursor thereof and PEA or a precursor thereof, spermidine or a precursor thereof and OEA or a precursor thereof, 1-MNA or a precursor thereof and PEA or a precursor thereof, 1-MNA or a precursor thereof and OEA or a precursor thereof, or PEA or a precursor thereof and OEA or a precursor thereof.

48. The method of any one of claims 24 to 45, wherein the method comprises administering three of the metabolites.

49. The method of claim 48, wherein the three metabolites are 1) sperimidine or a precursor thereof, 1-MNA or a precursor thereof, and PEA or a precursor thereof, 2) spermidine or a precursor thereof, 1-MNA or a precursor thereof, and OEA or a precursor thereof, 3) spermidine or a precursor thereof, PEA or a precursor thereof, and OEA or a precursor thereof, or 4) 1-MNA or a precursor thereof, PEA or a precursor thereof, and OEA or a precursor thereof.

50. The method of claims 24 to 45, wherein the method comprises administering all four of the metabolites.

51. The method of any one of claims 24 to 50, wherein the subject is a human.

52. A method extending lifespan, healthspan, healthy aging, altering biochemical pathways associated with the aging process or the treatment or prevention of age related diseases including but not limited to frailty, sarcopenia, dementia, Alzheimers disease, arthritis, and cancer in a subject in need thereof comprising administering to the subject a therapeutically effective amount of the composition of any one of claims 1-23.

53. The method of claim 52, wherein the subject is a human.

54. A method of increasing plasma cholesterol efflux ability in a subject in need thereof comprising administering to the subject a therapeutically effective amount of the composition of any one of claims 1-23.

55. The method of claim 54, wherein the subject is at risk for or has heart disease, stroke, arteral plaque formation or other cardiovascular disease risk factors.

56. The method of claim 54 or 55, wherein the subject is a human.
